SEQUENCE LISTING (35 sequences)

SEQ ID NO: 1 - DNA, 35 bp, Artificial
  oligonucleotide
taagaatgcggccgcgcgtggatggcgtctccagg

SEQ ID NO: 2 - DNA, 36 bp, Artificial
  oligonucleotide
gtttagtgaaccgtcagtcgctctgcggagaggctg

SEQ ID NO: 3 - DNA, 34 bp, Artificial
  oligonucleotide
ctgacggttcactaaacgagctctgcttatatag

SEQ ID NO: 4 - DNA, 34 bp, Artificial
  oligonucleotide
acgcgaattcactagttgttcctgcaatatctga

SEQ ID NO: 5 - DNA, 30 bp, Artificial
  oligonucleotide
ggacggaattcaatgctagctaagttaagg

SEQ ID NO: 6 - DNA, 41 bp, Artificial
  oligonucleotide
tatcaaatgcggccgcttttagcgagtttccttcttgtcag

SEQ ID NO: 7 - DNA, 31 bp, Artificial
  oligonucleotide
ataagaatgcggccgcaccagcacttggccg

SEQ ID NO: 8 - DNA, 30 bp, Artificial
  oligonucleotide
taagaatgcggccgcttacataacttacgg

SEQ ID NO: 9 - DNA, 39 bp, Artificial
  oligonucleotide
ggcggatccatatagatctgcgacagagactcttgcggg

SEQ ID NO: 10 - DNA, 35 bp, Artificial
  oligonucleotide
ccgcctcgagttattagcgagtttccttcttgtca

SEQ ID NO: 11 - DNA, 35 bp, Artificial
  oligonucleotide
gcggctcgagaacagcagggactttccacaagggg

SEQ ID NO: 12 - DNA, 38 bp, Artificial
  oligonucleotide
gggcgaattccccggatccctcgacctgcagctgcaaa

SEQ ID NO: 13 - DNA, 47 bp, Artificial
  oligonucleotide
ccgcgtcgacttactagttatcacaagagagtgagctcaagcccttg

SEQ ID NO: 14 - DNA, 31 bp, Artificial
  oligonucleotide
ggcggtcgacatgtctcattttataaaagaa

SEQ ID NO: 15 - DNA, 24 bp, Artificial
  oligonucleotide
gcgcctcgagcccctctccctccc

SEQ ID NO: 16 - DNA, 22 bp, Artificial
  oligonucleotide
gtctcttgttccatggttgtgg

SEQ ID NO: 17 - DNA, 24 bp, Artificial
  oligonucleotide
cgcgccatggtgagcaagggcgag

SEQ ID NO: 18 - DNA, 24 bp, Artificial
  oligonucleotide
ccgcctcgagttacttgtacagct

SEQ ID NO: 19 - DNA, 36 bp, Artificial
  oligonucleotide
ggatcgcggccgctgctagggattttcctgcttcgg

SEQ ID NO: 20 - DNA, 23 bp, Artificial
  oligonucleotide
ggcgcctgaacagggacttgaag

SEQ ID NO: 21 - DNA, 41 bp, Artificial
  oligonucleotide
ttttttctccatctcccactctatcttattaccccttcctg

SEQ ID NO: 22 - DNA, 29 bp, Artificial
  oligonucleotide
gagtgggagatggagaaaaaaatcactgg

SEQ ID NO: 23 - DNA, 31 bp, Artificial
  oligonucleotide
actagtgcatgcaggatccagacatgataag

SEQ ID NO: 24 - DNA, 27 bp, Artificial
  oligonucleotide
ctaaccgcaagaggccttcttaacatg

SEQ ID NO: 25 - DNA, 27 bp, Artificial
  oligonucleotide
ggagtcactctgcccagcaccggccca

SEQ ID NO: 26 - DNA, 24 bp, Artificial
  oligonucleotide
ggctgacaagaaggaaactcgcta

SEQ ID NO: 27 - DNA, 28 bp, Artificial
  oligonucleotide
ggagtcactctgcccagcaccggccaag

SEQ ID NO: 28 - DNA, 19 bp, Artificial
  oligonucleotide
atggaaaacccagctgaag

SEQ ID NO: 29 - DNA, 21 bp, Artificial
  oligonucleotide
cccagtacatgaccttatggg

SEQ ID NO: 30 - DNA, 20 bp, Artificial
  oligonucleotide
ccaaaaccgcatcaccatgg

SEQ ID NO: 31 - DNA, 20 bp, Artificial
  oligonucleotide
tcttccctgacaagacggag

SEQ ID NO: 32 - DNA, 275 bp, Artificial
  recombinant promoter
gctaaaagcggccgcttacataacttacggtaaatggcccgcctggctgaccgcccaacgacccccgcccattgacgtcaataatgacgtatgttcccatagtaacgccaatagggactttccattgacgtcaatgggtgtttgttttggcaccaaaatcaacgggactttccaaaatgtcgtaacaactccgccccattgacgcaaatgggcggtaggcgtgtacggtgggaggtctatataagcagagctcgtttagtgaaccgtcagtcgct

SEQ ID NO: 33 - DNA, 544 bp, Artificial
  recombinant promoter
gctaaaagcggccgcttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtcgct

SEQ ID NO: 34 - DNA, 274 bp, Artificial
  recombinant promoter
gctaaaagcggccgcttacataacttacggtaaatggcccgcctggctgaccgcccaacgacccccgcccattgacgtcaataatgacgtatgttcccatagtaacgccaatagggacttccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtcgct

SEQ ID NO: 35 - DNA, 351 bp, Artificial
  recombinant promoter
gctaaaagcggccgcttacataacttacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtcgct

(19) United States
(12) Patent Application Publication
(10) Pub. No.: US 2007/0065852 A1
(43) Pub. Date: Mar. 22, 2007

(54) HIV recombinant vaccine

(75) Inventors: Simon Wain-Hobson, Montigny-le-Bretonneux (FR); Philippe Blancou, Paris (FR); Nicole Chenciner, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Institut Pasteur

(21) Appl. No.: 11/512,315

(22) Filed: Aug. 30, 2006

Related U.S. Application Data
(63) Continuation of application No. 10/268,927, filed on Oct. 11, 2002, now abandoned.
(60) Provisional application No. 60/328,449, filed on Oct. 12, 2001.

(51) Int. Cl. C12Q 1/68; C12P 21/06

(52) U.S. Cl. 435/6; 435/69.1

(57) ABSTRACT

Reagents and methods for making and using HIV recombinant vaccines are disclosed.

### CROSS-REFERENCE TO RELATED APPLICATIONS

This is a continuation of application Ser. No. 10/268,927, filed Oct. 11, 2002, and claims the benefit of U.S. Provisional Application No. 60/328,449, filed Oct. 12, 2001, both of which are hereby incorporated herein by reference.

### BACKGROUND OF THE INVENTION

1. Field of the Invention

The invention is directed to the discovery that it is possible to severely attenuate lentiviral replication in vivo by changing promoter activity. The different U3 promoter/enhancer regions of wild type virus and cytomegalovirus result in differential replication in vivo. Despite feeble growth, the immune responses induced by recombinant viruses are capable of controlling viremia to an unprecedented degree.

2. Background

The macaque simian immunodeficiency virus (SIVmac) has been attenuated by a variety of genetic lesions in any of four loci and as such they do not encode a full complement of proteins. Highly attenuated simian immunodeficiency viruses (SIV) harbouring deletions in a variety of genes can elicit strong protection against intravenous challenge with pathogenic SIV strains (10, 11, 39). To date, they are the most efficient immunogens available. As more deletions were introduced the viral replication became more and more attenuated in vivo, sometimes inducing poor immune responses (11). An inverse relationship was found between the degree of attenuation and the degree of protection against homologous challenge (19). However, as these attenuated viruses persist and replicate some, notably the Anef viruses, can pick up further mutations in other sites and recover pathogenicity after a long term infection (14, 37). Furthermore they can recombine with the challenge virus (16, 22).

Deletions in various genes alter not only virus growth kinetics but also result in the loss of epitopes. SIV Δnef is a case in point. There are numerous publications linking the control of viremia to the early proteins Tat, Rev and Nef (1, 4, 28, 30). Therefore, the advantages of deleting Nef function are offset by loss of early epitopes. A number of live virus vaccines are attenuated by lesions in non-coding regions, the Sabin polio 3 vaccine strains being the most striking example (38). One of the most crucial attenuating lesions is a substitution in the 5′ non-coding internal ribosomal entry site, or IRES. Although the vaccine strain reverts to pathogenic strain within 4-5 days the virus is held in check by the immune responses.

Efficient transcription and replication of SIV can be achieved in the absence of NF-kB and Sp1 binding elements ex vivo (18) and can induce AIDS in rhesus monkeys in vivo (17). This result was due to a regulatory element located immediately upstream of NF-kB binding site that allows efficient viral replication in absence of the entire core enhancer region (32). By replacing the SIV enhancer promoter region by that of CMV-IE, a very similar replication profile on CEMx174 or PBMCs was obtained (18). By contrast, the virus was very attenuated in vivo even though it could replicate and establish a chronic infection contrarily to ΔNF-κB ΔSp1234 constructs (17). This virus retained the capacity to replicate in his host as proven by deletion analysis. First, these data show that CMV-IE promoter is able to overcome upstream regulatory element defined by Pohlmann et al. and, secondly, that variation in the pattern of protein expression by promoter can lead to drastic physiopathologic changes.

How the primate immunodeficiency viruses establish life long infection is still unclear, despite a wealth of studies. Certainly, the virus can remain transcriptionally silent in long lived memory T cells and evade immune surveillance (9). Virus can be recovered from these cells when they encounter the cognate antigen (7, 29). A test of this hypothesis would be the construction of a chimeric virus with a constitutive promoter leading to permanent presentation to cellular antiviral immunity. However, the promoter would have to be very strong for genomic RNA is spliced into more than 20 mRNA transcripts with a fraction of unspliced RNA being packaged.

Thus, there exists a need in the art for methods and reagents for using attenuated live virus vaccines to treat diseases caused by primate immunodeficiency viruses.

### SUMMARY OF THE INVENTION

The invention encompasses recombinant HIV and SIV viruses containing heterologous transcriptional regulatory elements in the U3 region of the virus. In particular embodiments, the recombinant virus has decreased replication in vivo and the virus has a protective effect when administered to a host.

The recombinant virus can have heterologous transcriptional regulatory elements replace the HIV region corresponding to the NFkB/Sp1/TATA Box/initiation region (−114 to +1) or corresponding to the NFkB/Sp1/TAR region (−114 to +93) of the SIVmac239 long terminal repeat.

The recombinant virus can have heterologous transcriptional regulatory elements inserted into a modified LTR generated by two PCR fragments formed with primers that correspond to the following sequences in SIV genome:

(SEQ ID NO:1)

5′-TAAGAATGCGGCCGCGCGTGGATGGCGTCTCCAGG
with

(SEQ ID NO:2)

5′-GTTTAGTGAACCGTCAGTCGCTCTGCGGAGAGGCTG
and

(SEQ ID NO:3)

5′-CTGACGGTTCACTAAACGAGCTCTGCTTATATAG
with

(SEQ ID NO:4)

5′-ACGCGAATTCACTAGTTGTTCCTGCAATATCTGA.

The recombinant HIV virus can have heterologous transcriptional regulatory elements inserted into a modified LTR generated by two PCR fragments formed with primers that correspond to the following sequences in SIV genome:

(SEQ ID NO:5)

5′-GGACGGAATTCAATGCTAGCTAAGTTAAGG
with

(SEQ ID NO:6)

5′-TATCAAATGCGGCCGCTTTTAGCGAGTTTCCTTCTTGTCAG
and

(SEQ ID NO:7)

5′-ATAAGAATGCGGCCGCACCAGCACTTGGCCG
with

(SEQ ID NO:4)

5′-ACGCGAATTCACTAGTTGTTCCTGCAATATCTGA.

The recombinant virus can be an SIV virus, SHIV virus, HIV-1 virus, or an HIV-2 virus. The recombinant virus can contain heterologous transcriptional regulatory elements replacing region −123 to +1 of HIV-1 virus or replacing region 190 to +1 of HIV-2 virus.

The recombinant virus can contain a promoter of a virus infecting human cells. In a particular embodiment, the virus contains a CMV-IE promoter from human cytomegalovirus.

The invention further encompasses expression vectors containing a nucleotide sequence of the recombinant viruses and cells containing these expression vectors.

The invention also encompasses processes for the production the recombinant viruses. In one embodiment, the process includes collecting peripheral blood, isolating the mononuclear cells in the blood, and infecting the mononuclear cells with the recombinant virus. In a further embodiment, the supernatant of the infected cells is collected.

The invention also encompasses immunogenic compositions containing the aforementioned recombinant viruses, vectors, and cells. In particular embodiments, the immunogenic compositions contain a pharmaceutically acceptable vehicle or carrier.

The invention also encompasses processes of measuring the immune response in a host comprising administering a recombinant virus and measuring the immune response to the virus.

In some embodiments, the host is infected with HI or SIV or SHIV. In another embodiment, the process includes boosting the immune system by modulating of the expression of the cytokines of the host.

### BRIEF DESCRIPTION OF THE FIGURES

FIG. 1 depicts the structure of SIVmac239/CMV-IE promoter chimeras. Central panel shows SIVmac239 LTR, while upper and lower panels show the structures of the chimeric SIVmegalo and SIVmegaloΔTAR. The positions of transcription factor binding motifs (for review see (27)), TAR sequences are shown.

FIG. 2 depicts replication kinetics of SIVmegaloΔTAR (A) and SIVmegalo (B) on CEMx174. Cells were infected with the same dose of virus for 5 million cells. Results of three separate experiments are given, verticals bars representing standard deviation.

FIG. 3 depicts rapid evolution of SIVmegalo promoter during replication on CEMx174 cells. (A) Genomic DNA was extracted from different time point and PCR was performed with primers within nef and 3′ to the TAR region. The SIVmegalo amplicon was 750 bp while that of SIVmac239 was 260 bp. (B) Sequences obtained after 15 or 60 days are reported as horizontal bars. Frequencies of sequences are reported on the right. A stock was derived after 2 months of culture of SIVmegalo on CEMx174 which gave rise to SIVΔMC. (C) Replication kinetics of SIVΔMC on CEMx174. Five million cells were infected by 1 ng of RT activity of SIVmac239, SIVmegalo, and SIVΔMC.

FIG. 4 depicts promoter activities of SIVmac239, SIVmegaloΔTAR, SIVmegalo, or SIVΔMC. (A) CEMx174 were transfected with chimeric LTR-CAT constructs with or without Tat. (B) CAT activity was measured 4 days later. SIVΔMC clone 61 is the promoter variant that predominated in a 60 day culture of SIVmegalo infected CEMx174 cells. The mean and standard deviation for three independent experiments are given.

FIG. 5 depicts replication kinetics of SIVmac 239 and SIVmegalo on macaque 93035 PBMCs. (A) Five million cells were infected by 1 ng of RT activity on 5×106 PBMCs. (B) Rapid evolution of SIVmegalo promoter during replication on PBMCs. The SIVmegalo amplicon was 750 bp while that of SIVmac239 was 260 bp. (C) Sequences obtained after 30 days of macaque 93035 PBMCs infection are reported as horizontal bars along with their frequencies on the right. The sequence denoted by a asterix is identical to the sequence found in lymph node after one hundred days of infection by SIVmegalo in macaque 93035 (see FIG. 7). (D) Replication kinetics of SIVmac239, SIVmegalo and SIVΔMC was assessed on PBMC of macaque 93033 and 93029. Five million PBMCs were infected by 1 ng of RT activity.

FIG. 6 depicts SIVmegalo and SIVΔMC infection in vivo. (A) Plasma viremia was determined by a bDNA assay. (B) Antibody titers are reported as reciprocal dilution of serum. A titer of one was arbitrarily given to undetectable SIV antibody. (C) PCR proviral detection in PBMCs (nested env V1 -V2, sensitivity 1-2 copies per reaction). Open circles are negative, filled circles are positive.

FIG. 7 A-B depicts evolved SIVmegalo promoters (SEQ ID NO:33). The major form at 60 days CEMx174 culture (SEQ ID NO:32) is typical of SIVΔMC. Two promoters from a culture on macaque PBMCs at 30 days are also shown (SEQ ID NO:34 and SEQ ID NO:35). The second promoter is identical to that found in the lymph node biopsies of animal 93035 at 100 days post-infection (SEQ ID NO:35). All ten LNMC sequences had the same 190 bp deletion. The 17, 18, 19 and 21 bp repeat are shown while known transcription factor binding sites are underlined.

FIG. 8 depicts expression of nef deleted IRES-GFP derivatives of SIVmac239 and SIVmegalo in CEMx174 and unstimulated macaque PBMCs (93035). A SIVΔNIG or SIVMIG clone 61 vectors contains the IRES of EMCV with florescent green protein as reporter in nef gene (A). These viruses were used to infect either CEMx174 or unstimulated PBMC from macaque 93035. (B). Expression was analysed by flow cytometry. The x axis designates cell number, while the y axis refers to fluorescence density of GFP. The mean value of GFP fluorescence per cell is indicated.

FIG. 9 depicts SwVmegalo (monkey 93035 and 93029) and SIVΔMC (monkey 94025) challenge in vivo. (A) Plasma viremia was determined by a bDNA assay. (B) Antibody titers are reported as reciprocal dilution of serum. A titer of one was arbitrarily given to undetectable SIV antibody. (C) PCR proviral detection in PBMCs (nested env V1-V2, sensitivity 1-2 copies per reaction). Open circles negative, filled circles positive.

### DETAILED DESCRIPTION OF THE INVENTION

The NF-kB/Sp1 region (−114 to +1) or the NF-kB/Sp1/TAR region (−114 to +93) of the SIVmac239 long terminal repeat have been replaced by the powerful immediate early promoter (−525 to +1) from human cytomegalovirus (CMV-IE). Of the two viruses SIVmegalo and SIVmegaloΔTAR respectively, only the former grew at all well on CEMx174 T cells, albeit delayed a few days compared to SIVmac239. During culture, the CMV-IE promoter proved unstable. However, a genetically stable derivative stock encoding a 272 bp deletion in CMV promoter was obtained after 60 days of culture on CEMx174. This stock, SIVΔMC, grew as well as parental 239 virus on CEMx174. When inoculated into rhesus macaques, both SIVmegalo and SIVΔMC showed highly controlled viremia during primary infection and persistent infection. After primary infection, plasma viremia was invariably below the threshold of detection and proviral DNA was only intermittently recovered from peripheral blood mononuclear cells. These findings show that it is possible to severely attenuate SIV replication in vivo by changing promoter activity. The different U3 promoter/enhancer regions of wild type and megalo virus result in differential replication in vivo. This difference might be related to the in vitro delay kinetics of replication on PBMCs.

While SIVmegalo and SIVΔMC grew well ex vivo, SIVmegaloΔTAR replication was feeble. Although the CMV-IE promoter is widely considered to be one of the strongest promoters currently used, indeed it has been used to drive expression of the SIV genome in the context of DNA vaccination (2, 13), it is insufficient alone to drive efficient SIV viral replication. Perhaps this relates to the fact that a single RNA transcript is spliced into at least 20 different mRNAs with a further fraction dimerising and thus being translationally inactive. With the powerful Tat/TAR transactivation system, the problem would appear to be overcome.

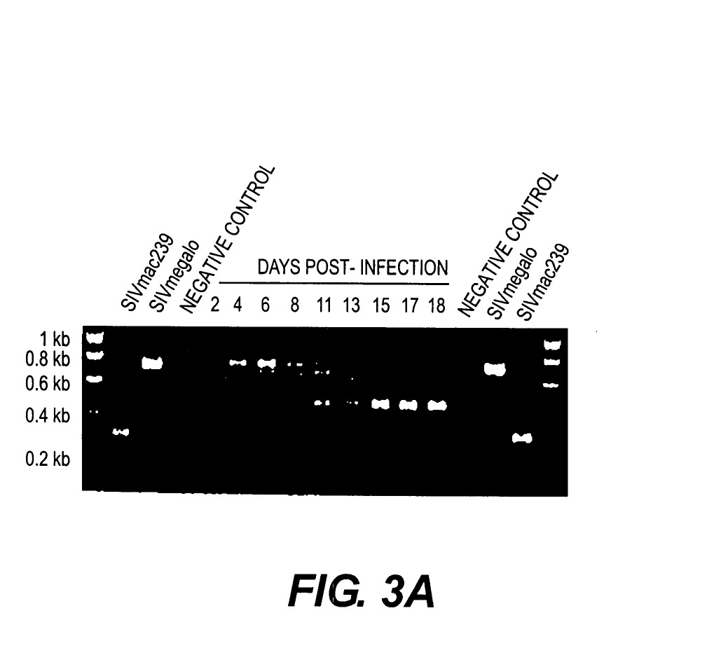

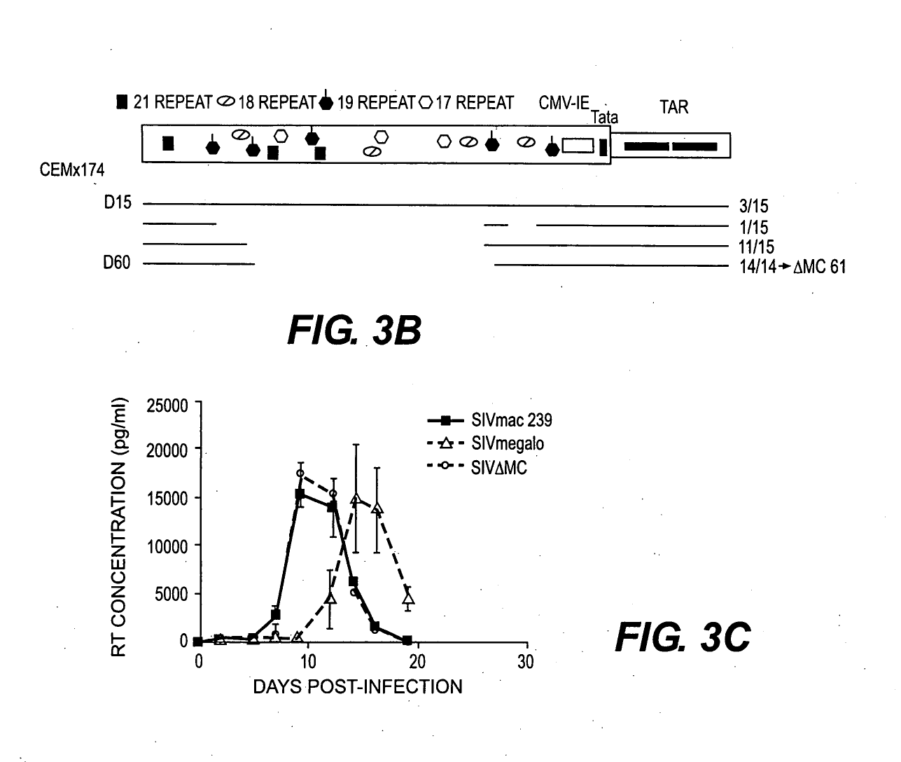

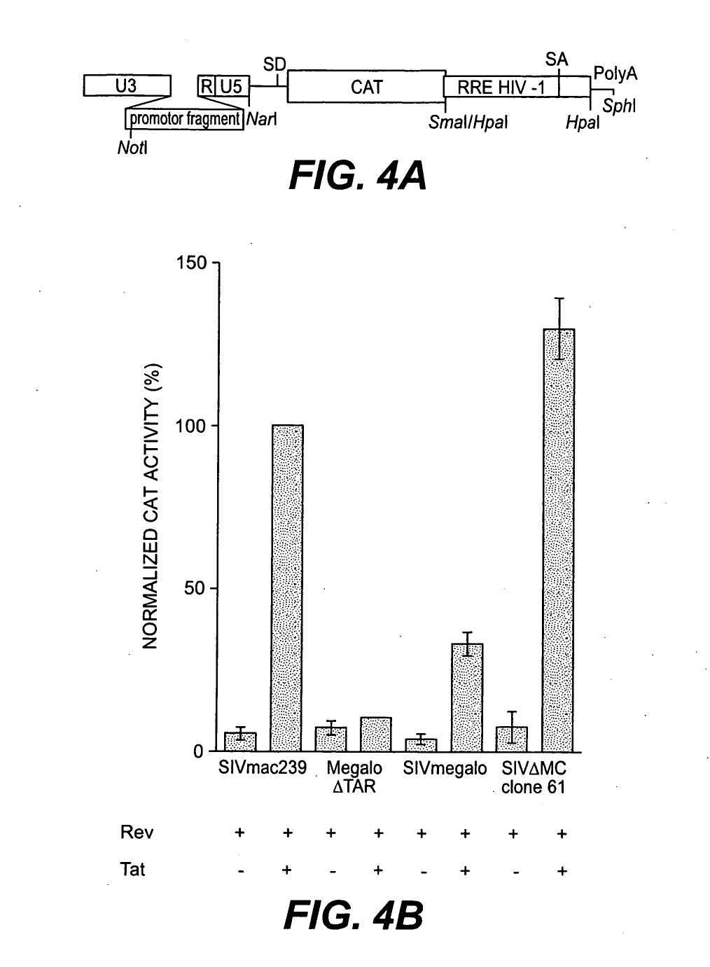

The CMV-IE promoter was not well adapted to the SIV scaffold for it grew initially slowly. When replication took off, it was accompanied by deletions in the promoter distal regulatory region between −450 to −200 bp. Once this region deleted in vitro, the mutant virus, termed SIVΔMC, acquired similar kinetics to wild type virus on CEMx174 cell line and on PBMC. The deletions presumably resulted in enhanced transcription and replication (burst size) resulting in their outgrowing other variants, something that was confirmed for deleted clone promoters in the CAT assay (FIG. 4B). A genetically stable virus stock (FIG. 3C) was derived from a 60 days CEMx174 culture. The SIVΔMC stock harboured a deletion resulted in the loss of the three 17 bp repeats, one 19 bp repeats, two 18 bp repeats and two 21 bp repeats, which encode 8 transcription factors motifs in total. Analogous deletions in the CMV-IE promoter have been made experimentally and have been shown to augment transcription in transfection assays, so there is general concordance (36). The transcriptional improvement is probably due to the rapprochement of regulatory elements, which act as an enhancer. Similarly, clones derived from lymph nodes of SIVmegalo infected monkey are deleted in a manner that do not affect enhancer/promoter activity (36). Thus, it seems that maximal CMV-IE activity is essential for viral replication. As the HIV/SIV RT is very prone to making deletions especially between homologous sequences (8, 24, 31), the rapidity with which they may be detected ex vivo or in vivo is understandable, particularly if there is a selective advantage.

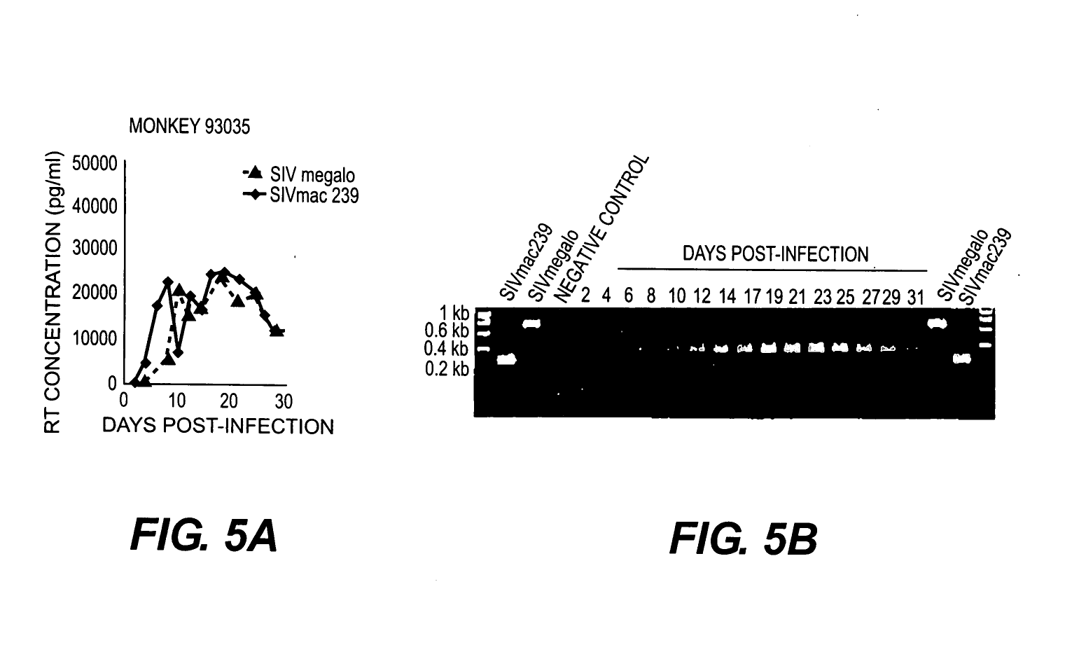

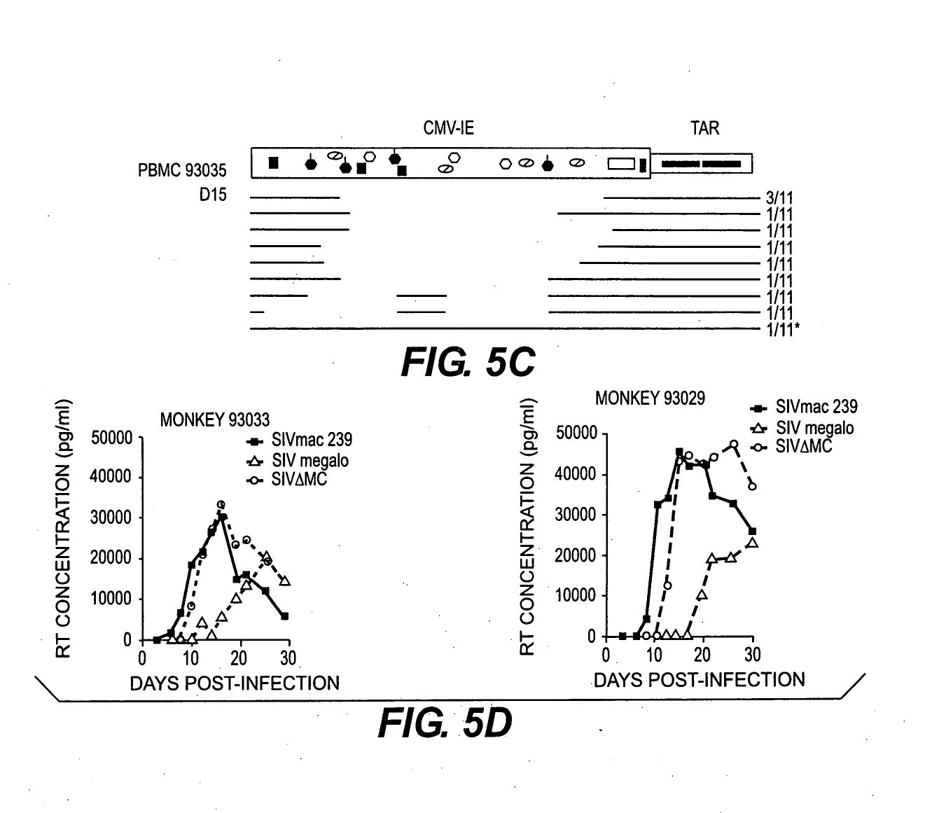

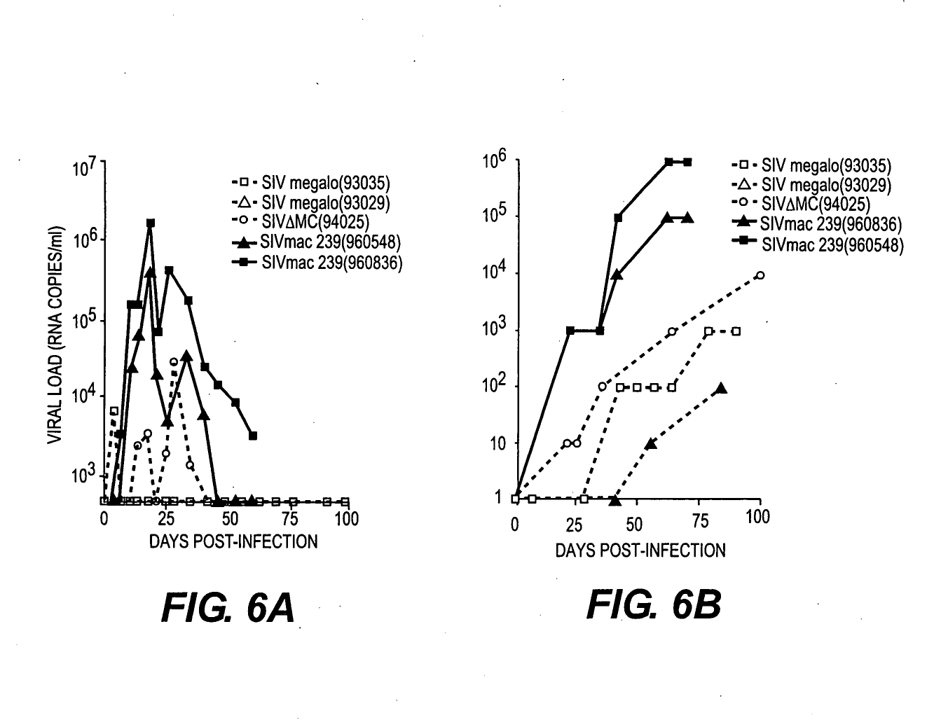

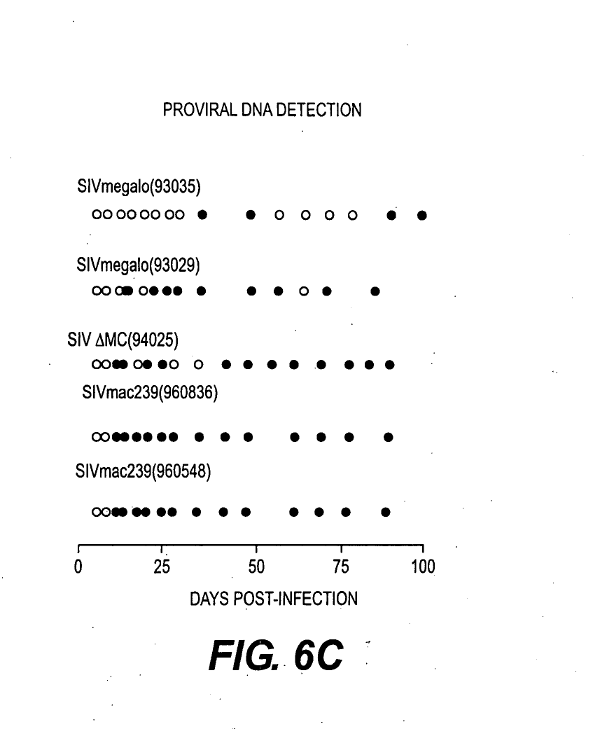

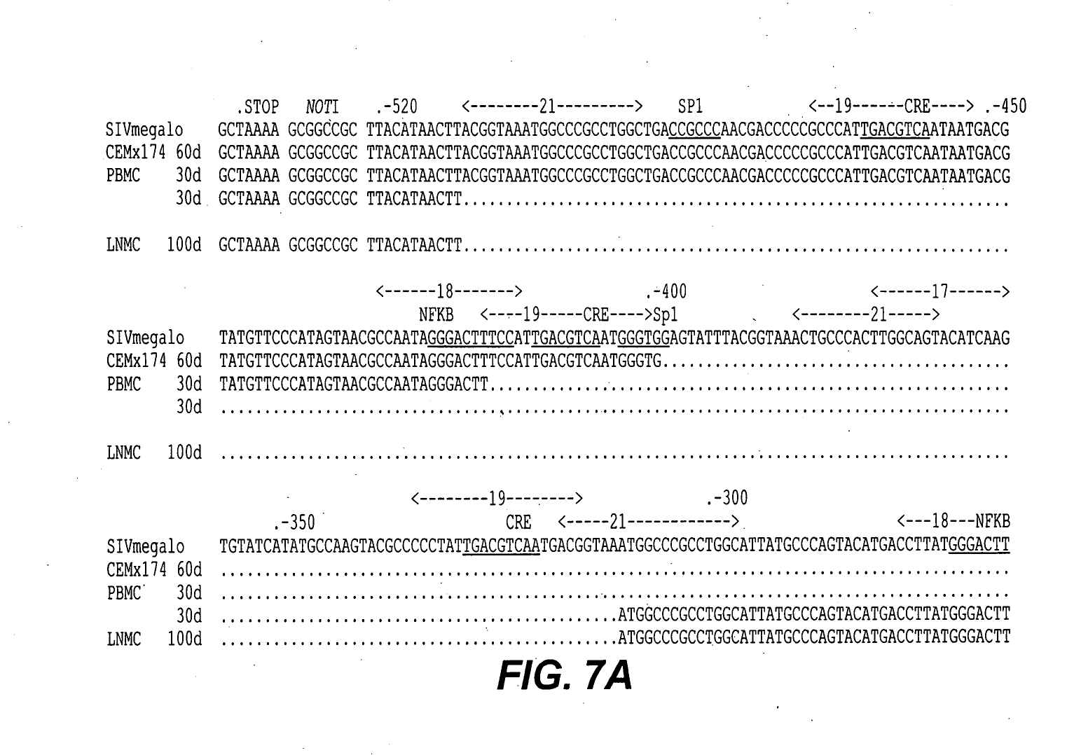

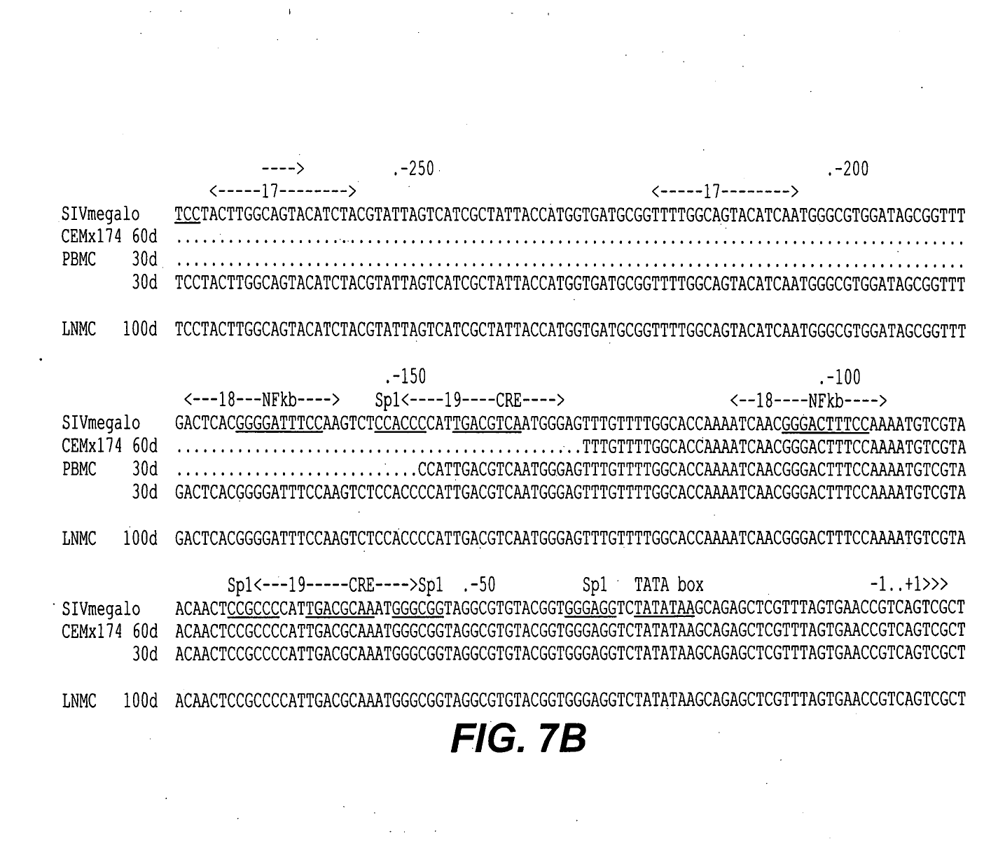

When inoculated into rhesus macaques, SIVmegalo grew very poorly, so much so that there was only one positive serum RNA sample between the two animals. Despite this, SIVmegalo infection established itself, since virus could be occasionally detected in PBMCs out to 100 days. The poor replication of SIVmegalo was reflected in the low antibody titres (FIG. 6B) which is a feature of highly attenuated SIVmac239 constructs such as Δvif (11). The CMV promoter readily accumulated deletions during in vitro cultures on PBMCs of macaque 93035 (FIG. 5), one minor form was identical to the major viral form obtained in LNMC of macaque 93035 after one hundred days of SIVmegalo infection (FIG. 7). The structure of promoter at 100 days was almost identical to a construct dlNdeI which functioned as well as, but no better than, the undeleted promoter in transient transfection assays (36).

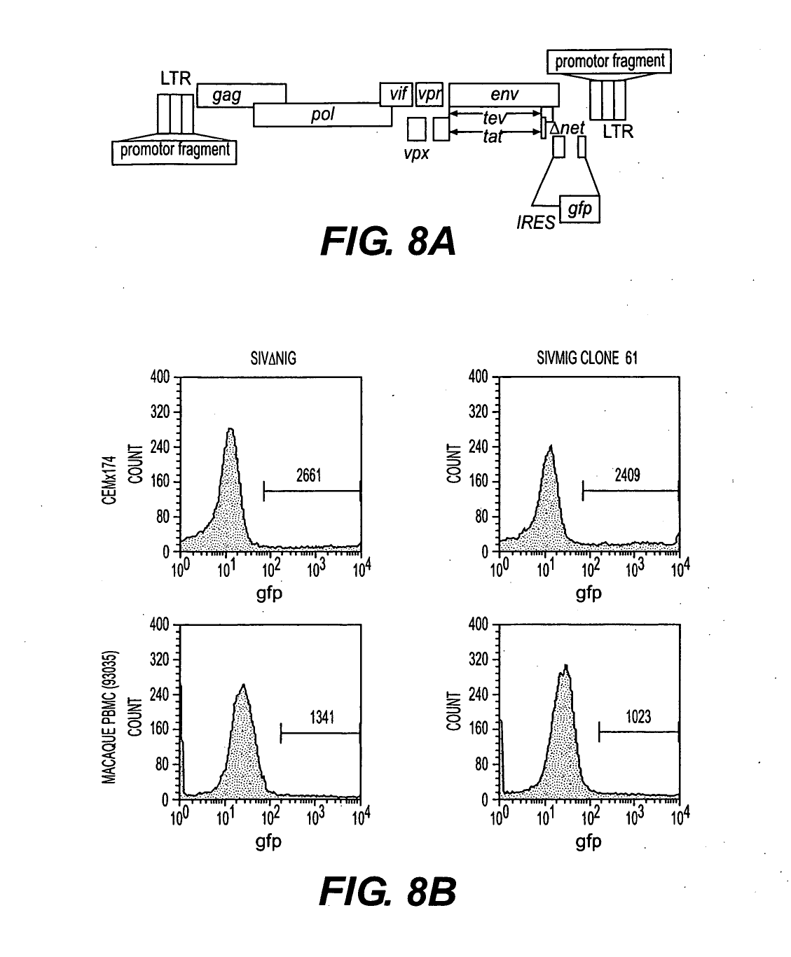

A similar situation pertained to SIVΔMC. In contrast to what might have been anticipated from its properties in vitro, SIVΔMC also grew poorly in vivo. Primary viremia was higher and antibody titre appeared earlier than for SIVmegalo indicative of greater replication, while SIV proviral DNA could be amplified more frequently for SIVΔMC than SIVmegalo (13/17 attempts versus 10/15 or 4/16, FIG. 6C). Be that as it may, the magnitude of primary viremia was some 2-3 logs down on parental SIVmac239. Given that SIVmac239 and SIVΔMC encode a full set of proteins the difference must lie in differential proviral transcription in vivo. Nef-deleted IRES-eGFP derivatives of both SIVmegalo and SIVmac239 failed to show any difference in eGFP expression on non-stimulated macaque PBMCs (FIG. 8C).

SIVmegalo and SIVΔMC grow very poorly in vivo. The level of viremia is very low by any standards. This means that the virus is infecting only a very small fraction of CD4 T lymphocytes. Independent confirmation of this are the low antibody titres in the three animals. Given that the virulence of a SIV infection is related to the replicative capacity of the virus, low viremia is a prerequisite for a live attenuated vaccine (Johnson et al., 1999).

Despite feeble growth, the immune responses induced are capable of controlling viremia to an unprecedented degree. In the naive animals peak viremia levels of 10⁶-10⁷ were noted. For the SIVmegallo and SIVΔMC inoculated animals, viremia was <400 copies/ml, the cut-off of the bDNA test. However recovery of challenge virus LTR sequences means that the virus took. In fact this is the outcome of all SIV vaccination/challenge studies published to date and concurs with the notion that vaccination in general rarely confers sterilizing immunity but rather prevents disease.

Yet in comparison to other vaccine studies using DNA and vaccinia based methods, challenge is invariably accompanied by a peak of plasma viremia between 1-3 weeks post challenge. The titres vary with the challenge virus and the animal, but can attain titres of 10⁵-10⁹ per ml (Amara et al., 2001). They then decayed to a set point which again varies but can be typically between undetectable (i.e. <100-400 copies/ml) to 10⁴/ml. Out to 2 months post challenge, plasma viremia was undetectable.

Discrepancies between ex vivo and in vivo have previously been noted and are typified by SIVmac239Δnef (10). Yet, given the lesion in nef, it could be argued that it influences the life cycle in vivo. As SIV replication depends on the relative dynamics of local replication with respect to control by anti-viral cellular immunity being played out over a matter of hours (P. Blancou, N. Chenciner, M. C. Cumont, S. Wain-Hobson, B. Hurtrel, submitted for publication), lower overall replication favours control by the immune system. Similar findings have been noted for a variety of attenuated SIV constructs bearing numerous gene deletions. In this context SIVmegalo and SIVΔMC are comparable to SIVmac239Δ4 which harbours deletions in vpx, vpr, nef and the overlapping U3/nef region of the LTR (11). This virus was estimated to be attenuated some 1000 fold and even offered partial protection to rectal challenge. However, all four animals failed to protect against challenge by the intravenous route (19).

There are precedents for the chimeric HIV and SIVs with the CMV-IE promoter. Chang et al. made three constructs in a HIV-1 background (6). Recombinants CMV-EE(a) and CMV-EE(b) encoded fragments from −535 to −37 and −535 to +1 respectively, both of which carried the −405 and −135 deletion in the enhancer region (6). The third construct, CMV-IE(a)/TATA, carried a shorter promoter fragment from −229 to -37. After a delay, CMV-IE(b) and CMV-E(a) replicated as well as the parental HIV-1 virus. Surprisingly, the CMV-IE(a)/TATA, which most closely resembles the present SIVΔMC construct, grew only on AA2 cells and not H9 or CEM cells.

Guan et al engineered the same CMV-E promoter into a SIVmac239 background along with a deletion in the nef gene (virus SIVmac239 Δnef-CMV)(15). The virus grew reasonably well on a variety of cell lines. As promoter stability was not checked, it is difficult to compare SIVmac239 Δnef-CMV with SIVΔMC.

SIV may be attenuated by merely altering the U3 enhancer/promoter region, which in turn shows that there are no immunosuppressive proteins per se. In this respect, SIVmegalo parallels attenuated Sabin polio 3 virus strains, which bear a crucial substitution in the 5′ non-coding IRES structure (38). Despite the rapid reversion of the lesion as little as 4-5 days post vaccination, the wild type virus is held in check by the immune system. Being a lifelong infection, reversion of retroviral lesions is more problematic.

Although there are numerous papers, the field of attenuated SIV vaccines was championed and remains dominated by the group of Ronald C. Desrosiers. Their idea has been to attenuate the virus by making deletions within the different SIV genes. If the deletions are sufficiently large, greater than 20 bases or more, the chance of the virus reverting in the same locus is nil. Among all their constructs, they find that attenuation follows the order SIVΔvpr>SIVΔvpx>SIVΔvpxΔvpr˜SIVΔnef>SIVΔvprΔnef□US>SIVΔvpxΔnefΔUS>SIVΔvpxΔvprΔnef□US>SIVΔvif>SIVΔvifΔvpxΔvprΔnefΔUS (see Table 1, (Desrosiers et al., 1998), ΔUS refers to a deletion in the U3 region of the LTR that overlaps the 3′ portion of the nef gene). To simplify description, we will use the abbreviations Desrosiers et al. gave to the viruses notably SIVΔ3 for SIVΔvprΔnefΔUS, SIVΔ3x for SIVΔvpxΔnefΔUS and SIVΔ4 for SIVΔvpxΔvprΔnefΔUS.

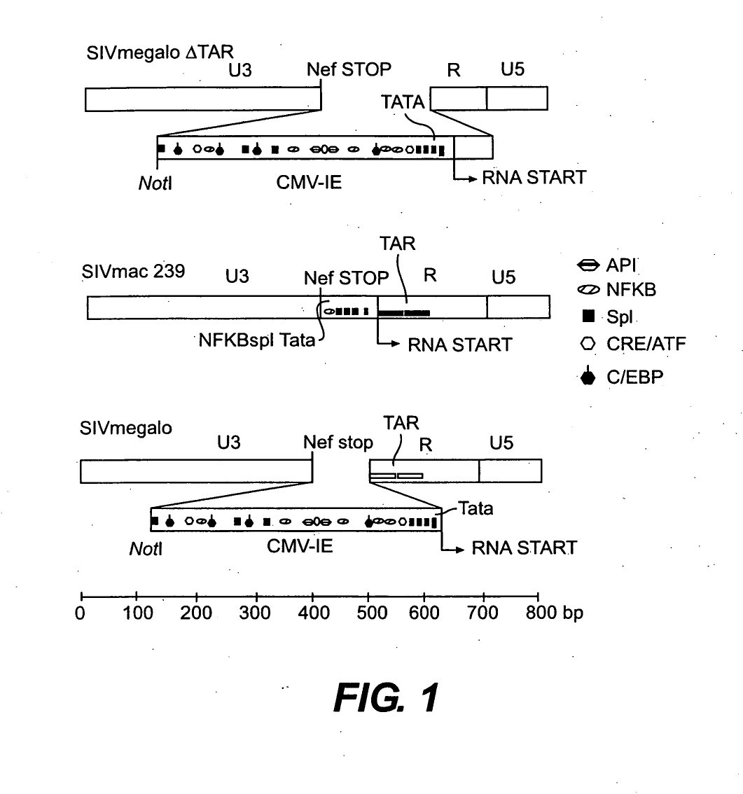

SIVmegalo and SIVΔMC show peak viremia comparable to SIVΔ4. When four macaques vaccinated by the SIVΔ4 virus were challenged by 10 animal infectious doses of uncloned SIVmac251 via the intravenous route, all four animals showed rapid breakthrough of the challenge virus. The level of cell-associated virus in the periphery (FIG. 3, (Desrosiers et al., 1998)) was comparable to that found for unvaccinated animals (FIG. 1D, (Desrosiers et al., 1998)).

By contrast, SIVmegalo and SIVΔMC protect against the equivalent of 2000 animal infectious doses of SIVmac239. These results are better than anything else published to date.

Two possible explanations, which are not mutually exclusive, of why low levels of SIV replication induce such robust immune responses ideas are:

1) Of all the attenuated viruses SIV made to date, only SIVmegalo and SIVΔMC encode a complete set of proteins. Many attenuated virus have deletions in the nef gene which produces the highly immunogenic protein, Nef. This gene is expressed early on in infection, at a time when virion assembly has not yet started. Hence, good cellular immunity to Nef and the other early gene proteins, Tat and Rev, might be prerequisites for efficient vaccination.

2) As SIV preys on the very CD4 T cells needed to induce good immunity, the anti-SIV CD4 T lymphocytes, low levels of replication allow the generation of robust immunity with little loss of these crucial T cells.

HIV-1 or HIV-2 derivatives with CMV-IE promoters, or any heterologous promoter, whether being of viral or eukaryotic origin, that results in highly reduced replication in vivo, can be used as live attenuated HIV virus vaccines. An advantage of these viruses over others is their complete complement of proteins and their low replication properties in primary infection.

Derivatives of such HIV-1 and HIV-2 promoter exchanged viruses with deletions within the open reading frames, for example vif, vpr, nef can be constructed to attenuate further the virus in a manner already described for SIV. The LTR could be redesigned so that nef and LTR no longer overlap. This would provide a vector in which the so called negative regulatory element (NRE) sequences can no longer act in cis on the endogenous or exogenous promoters that will be used, a phenomenon that has been already noted in lentiviral vectorology

Recently the group of Mark Wainberg at the University of Toronto, Canada, made a derivative of SIV which resembles the SIVmegalo construct (Guan et al., 2001). Their virus, termed SIVmac239Δnef-CMV, contained a deleted nef gene as its name implies (FIG. 1B, (Guan et al., 2001)). It appears that virtually all of the SIV U3 promoter region was deleted and replaced by the CMV-IE promoter. The resulting virus grew well on the human T cell line CEMx174. The growth properties of the virus on macaque PBMCs was not described although a derivative of the virus with inactivating mutations in the tat gene grew very poorly indeed with peak p27 antigenemia not reaching more than 0.1 ng/ml, which is ˜2.5 logs less than wild type SIVmac239 (FIG. 11A, (Guan et al., 2001)). Guan et al. described a large number of SIV derivatives none of which grew well in monkey PBMCs. No in vivo work was reported

By contrast SIVmegalo grows well on monkey PBMCs after a delay of 5-7 days with respect to SIVmac239. SIVΔMC grows almost as well as SIVmac239 with only three days delayed on macaque PBMCs.

The invention encompasses recombinant HIV and SIV viruses containing heterologous transcriptional regulatory elements in the U3 region of the virus. In particluar embodiments, the recombinant virus has decreased replication in vivo and the virus has a protective effect when administered to a host.

In one embodiment, the invention encompasses a recombinant SIV or HIV virus in which sequences in the natural transcriptional regulatory elements in the U3 region of the virus have been replaced by sequences encoding heterologous transcriptional regulatory elements.

In another embodiment, recombinant SIV or HIV is purified. In one embodiment, purified SIV or HIV is free of cells. In another embodiment, purified SIV or HIV is purified on a gradient or by pelletting by centrifugation.

A recombinant SIV or HIV virus is one that has been genetically altered to recombine a naturally occurring nucleic acid sequences of the virus with at least one non-naturally occurring nucleic acid sequence. Many molecular biological methods known in the art including PCR can be used to generate a recombinant HIV or SIV virus.

In one embodiment, the HIV virus is an HIV-1 virus. In another embodiment, the HIV virus is an HIV-2 virus. In another embodiment, the virus is a SHIV virus. A SHIV virus is an SIV virus in which a part of the HIV genome has been integrated.

The“replaced sequences” or“replaced region” refers to those bases that are deleted with respect to a naturally occurring wild-type purified SIV or HIV virus. In one embodiment, the naturally occurring wild-type purified SIV virus is wild-type SIVmac239. In another embodiment, the naturally occurring wild-type purified HIV is HIV-1BRU. In another embodiment, the naturally occurring wild-type purified HIV is HIV-2ROD.

The replaced sequences or replaced region can be as few as 25 bases, preferably at least 30, 40, 50, 60, 70, 80, or 90 bases, and more preferably at least 100, 120, 150, 200, 250, 300, 400, or 500 bases. Replaced regions of less than 500, 400, 300, 250, 200, 150, 120, 100, 90, 80, 70, 60, 50, 40, 30, and 25 bases are also preferred. Particularly preferred are regions of 25-500 bases, 90-100 bases, and all other ranges of bases that can be extrapolated from the above-mentioned range endpoints.

In one embodiment, the replaced sequences are bases −123 to +1 relative to the transcriptional start site of genomic RNA of an HIV-1 virus. In another embodiment, the replaced sequences are bases −190 to +1 relative to the transcriptional start site of genomic RNA of an HIV-2 virus. In another embodiment, the replaced sequences are bases −114 to +1 relative to the transcriptional start site of genomic RNA of SIVmac239. In another embodiment, the replaced sequences are bases −114 to +93 relative to the transcriptional start site of genomic RNA of SIVmac239.

In another embodiment, the replaced sequences correspond to bases −114 to +1 relative to the transcriptional start site of genomic RNA of SIVmac239, or bases −114 to +93 relative to the transcriptional start site of genomic RNA of SIVmac239, but are from a virus that is homologous to this virus. In this context, “corresponds to” refers to those sequences of another virus that maximally align by comparison of sequence homology with this region of SIVmac239.

Likewise, “corresponds to” can be used in reference to other HIV and SIV strains. For example, sequences may correspond to bases −190 to +1 relative to the transcriptional start site of genomic RNA of HIV-2ROD or bases −123 to +1 relative to the transcriptional start site of genomic RNA of HIV-1BRU. Sequences that correspond to a given sequence are preferably 30% identical, more preferably 50%, 60%, or 70% identical, and most preferably 80%, 90%, 95%, or 99% identical in nucleotide sequence.

The “replacement sequences” or “replacement region” refers to those bases that are inserted with respect to a naturally occurring wild-type purified SIV or HIV virus. In one embodiment, the naturally occurring wild-type purified SIV virus is wild-type SIVmac239. In another embodiment, the naturally occurring wild-type purified HIV is HIV-1BRU. In another embodiment, the naturally occurring wild-type purified HIV is HIV-2ROD.

The replacement sequences or replacement region can be can be as few as 25 bases, preferably at least 30, 40, 50, 60, 70, 80, or 90 bases, and more preferably at least 100, 120, 150, 200, 250, 300, 400, or 500 bases. Replaced regions of less than 500, 400, 300, 250, 200, 150, 120, 100, 90, 80, 70, 60, 50, 40, 30, and 25 bases are also preferred. Particularly preferred are regions of 25-500 bases, 90-100 bases, and all other ranges of bases that can be extrapolated from the above-mentioned range endpoints.

Heterologous transcriptional regulatory elements include heterologous promoter or heterologous enhancer elements. A heterologous promoter or heterologous enhancer is a promoter or enhancer that is operably linked to a nucleic acid sequence that it is not normally linked to in nature. The heterologous promoter or enhancer can be any eukaryotic, prokaryotic, synthetic, or viral promoter or enhancer. In one embodiment, the heterologous transcriptional regulatory element is a eukaryotic promoter. In another embodiment, the heterologous promoter is a viral promoter. In another embodiment, the viral promoter is from a virus that infects human cells. In another embodiment, the heterologous promoter is a cytomegalovirus immediate early promoter (CMV-IE).

In some embodiments the recombinant virus contains a CMV-IE promoter/enhancer having deletions in the −420 to −130 region. In some embodiments, the virus has transcriptional regulatory elements having a sequence shown in FIG. 7. In other embodiments, a recombinant HIV-1 virus contains a CMV-IE promoter having a deletion of the −420 to −130 region depicted in FIG. 7.

In one embodiment, the recombinant virus replicates poorly in a host. In one embodiment, the recombinant virus replicates to wild-type titers in PBMCs, but grows to a peak primary viremia titer in a host of at least 1 log less than the wild-type virus. In another embodiment, the recombinant virus replicates to wild-type titers in PBMCs, but grows to a peak primary viremia titer in a host of at least 2 logs less than the wild-type virus. In another embodiment, the recombinant virus replicates to wild-type titers in PBMCs, but grows to a peak primary viremia titer in a host of at least 3 logs less than the wild-type virus. In one embodiment, the recombinant virus replicates to at least 0.5, 0.3, or 0.1 of wild-type titers in PBMCs.

In another embodiment, the recombinant virus is immunogenic. An immunogenic composition containing the recombinant virus is encompassed by the invention. The immunogenic composition can contain an pharmaceutically acceptable carrier or vehicle. Immunogenic compositions can also contain expression vectors of the invention, cells containing the expression vectors or viruses of the invention, particularly infected mononuclear cells.

In another embodiment, an antiviral antibody response is detectable 20 days after infection of the host with the recombinant virus. In other embodiments, an antiviral antibody response is detectable 30, 40, 50, 75, or 100 days after infection of the host with the recombinant virus. In another embodiment, the antiviral antibody response is at least 1 log less, at least 2 logs less, or at least 3 logs less than that generated by the wild-type virus at a particular timepoint post-infection. In other embodiments, the timepoint is 20, 30, 40, 50, 75, or 100 days after infection.

In another embodiment, the recombinant virus has a protective effect when administered to a host. That a virus has a “protective effect when administered to a host,” means that the host has no detectable plasma viremia (i.e. <400 copies/ml) at all timepoints out to two months post-challenge with a wild-type virus.

In one embodiment, the recombinant SIV or HIV virus contains all of the genes of a wild-type virus. In another embodiment, the recombinant virus is deleted for at least part of the nef gene, the vif gene, the vpr gene, the vpx gene or the vpu gene, individually, or in any combination. For example, the recombinant virus may be deleted for at least part of vpx and vpr, vpr and nef, vpx and nef, vpx and vpr and nef, or vif and vpx and vpr and nef The recombinant virus may also be deleted at least part of the tat or rev gene.

The invention further encompasses expression vectors containing nucleic acid sequences of recombinant HIV or SIV viruses. The invention also encompasses cells containing expression vectors containing nucleic acid sequences of the recombinant HIV or SIV viruses and cells containing recombinant HIV or SIV viruses.

The invention further encompasses processes for the production of SIV or HIV. In one embodiment, the virus is produced by infecting mononuclear cells with recombinant HIV or SIV. In another embodiment, SIV or HIV is isolated by collecting cell supernatant from infected cells. In another embodiment, mononuclear cells are isolated from peripheral blood. In another embodiment, the peripheral blood is human blood.

The recombinant HIV and SIV can be formulated into pharmaceutical compositions, which can be delivered to a subject, so as to allow production of attenuated virus. Pharmaceutical compositions comprise sufficient virions that allows the recipient to produce an immunogenic response against the administered virus. Particularly, 1-2000 TCID₅₀ (tissue culture infections dose) of the virus are used. More particularly, 1-200 TCID₅₀ of the virus are used. In a particular embodiment, 200 TCID₅₀ of the virus are used.

The compositions may be administered alone or in combination with at least one other agent, such as stabilizing compound, which may be administered in any sterile, biocompatible pharmaceutical carrier, including, but not limited to, saline, buffered saline, dextrose, and water.

The compositions may be administered to a patient alone, or in combination with other agents, clotting factors or factor precursors, drugs or hormones. In some embodiments, the pharmaceutical compositions also contain a pharmaceutically acceptable excipient. Such excipients include any pharmaceutical agent that does not itself induce an immune response harmful to the individual receiving the composition, and which may be administered without undue toxicity.

Pharmaceutically acceptable excipients include, but are not limited to, liquids such as water, saline, glycerol, sugars and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients that could be used in this invention is available in Remington's Pharmaceutical Sciences (Mack Pub. Co., 18th Edition, Easton, Pa. [1990]).

Pharmaceutical formulations suitable for administration may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

It is intended that the dosage treatment and regimen used with the present invention will vary, depending upon the subject and the preparation to be used. Thus, the dosage treatment may be a single dose schedule or a multiple dose schedule. Moreover, the subject may be administered as many doses as appropriate to achieve or maintain the desired immunogenic response.

Direct delivery of the pharmaceutical compositions in vivo may be accomplished via injection using a conventional syringe. In some embodiments, the compositions are administered intravenously. In other embodiments, delivery is intramucosally, eg., rectally or vaginally.

Recombinant viruses can be used to treat either patients infected with HIV or those uninfected by administering the recombinant virus to the patient, measuring the immune response, and optionally boosting the immune system by modulating the expression of cytokines of the patient.

Recombinant viruses can be used to induce an immune response in a primate host. An immunogenic composition containing the recombinant virus can be introduced into the host. In a particular embodiment, the recombinant virus contains a heterologous CMV-IE promoter/enhancer sequence replacing part of the U3 sequence of the lentvirus, which causes the virus to replicate poorly in vivo, while inducing an strong antibody response.

SIV can be used in an animal model for the development of recombinant HIV vectors. In a particular model, an SIV containing a heterologous promoter is used in rhesus macaques to select for corresponding regions of HIV and to select for heterologous promoters for attenuated recombinant virus production. As part of this selection, recombinant viruses can be passaged in culture, particularly in PBMC, or in vivo, and the resultant viruses analysed.

The invention also encompasses a process of selection of an animal model for testing an immunogenic composition according to the invention. A recombinant SIV or SHIV virus of the invention can be used in an animal model for vaccination, and immunogenic response and viremia can be measured. Results with the animal model can be used to predict results with HIV viruses having similar heterologous transcriptional regulatory elements.

The specification is most thoroughly understood in light of the teachings of the references cited within the specification which are hereby incorporated by reference. The embodiments within the specification and the examples provide an illustration of embodiments of the invention and should not be construed to limit the scope of the invention. The skilled artisan readily recognizes that many other embodiments are encompassed by the invention.

### EXAMPLE 1

### Construction of Chimeric Viruses

Two derivatives of SIVmac239, SIVmegalo and SIVmegaloΔTAR constructs, were made by first deleting SIV U3 promoter sequences between the nef stop codon and the SIV transcription start (−114 to +1) or from −114 to +93, just 3′ to the double TAR motifs. The cytomegalovirus immediate early promoter (CMV-IE) was cloned in its place. The two chimeras were called SIVmegalo and SIVmegaloΔTAR.

The wild type SIVmac239 was available as two plasmids p239SpSp5′ and p239SpE3′ which contain the 5′ and 3′ halves of the genome, respectively (20, 34). The 3′ plasmid was unmodified and hence contains the nef stop signal which was shown to revert rapidly after in vivo infection (21). For the SIVmegalo and ΔTAR constructions, both half plasmids were modified. For the SIVmegaloΔTAR construction the modified LTR was first generated from two PCR fragments using primers: 5′ GGACG GAATTCAAT GCTAGCTAAGTTAAGG (SEQ ID NO:5) with 5′ TATCAAAT GCGGCCGCTTTTAGCGAGTTTCCTTCTTGTCAG (SEQ ID NO:6) and 5′ ATAAGAAT GCGGCCGCACCAGCACTTGGCCG (SEQ ID NO:7) with 5′ ACGC GAATTCACTAGT TGTTCCTGCAATATCTGA (SEQ ID NO:4). EcoRI, SpeI, NotI, and NheI restriction sites are underlined. These two PCR products were subcloned. For cloning in the 5′ half plasmid the products were cut with EcoRINotI and NotI/SpeI respectively, gel purified and ligated into p239SpSp5′. For the 3′ half plasmid the products were cut with NheI/NotI and NotI/EcoRI respectively, gel purified and ligated into p239SpE3′. The 532 bp CMV-IE promoter was amplified from a pCMV-CAT plasmid using primers containing flanking NotI sites i.e. 5′ TAAGAAT GCGGCCGCGCGTGGATGGCGTCTCCAGG (SEQ ID NO:1) and 5′ TAAGAAT GCGGCCGCTTACATAACTTACGG (SEQ ID NO:8). This fragment was then subcloned into the previous constructions at the NotI site. The two half plasmids were called pMT-5 and pMT-3.

For the SIVmegalo construction, two PCR fragments were generated using respectively the SIVmegaloΔTAR construction and CMV-IE promoter with the following primers: 5′ TAAGAAT GCGGCCGCGCGTGGATGGCGTCTCCAGG (SEQ ID NO:1) with 5′ GTTTAG TGAACCGTCAGTCGCTCTGCGGAGAGGCTG (SEQ ID NO:2) and 5′ CTG ACGGTTCACTAAACGAGCTCTGCTTATATAG (SEQ ID NO:3) with 5′ ACGC GAATTCACTAGTTGTTCCTGCAATATCTGA (SEQ ID NO:4) (NotI and EcoRI sites underlined). PCR products were purified with primer purification kit (Quiagen) and annealed in PCR mix without primer for 5 cycles. External primers were then added for 30 more cycles. Annealed PCR products were cloned, double digested with NotI and NarI and the resulting fragment were gel purified and introduced in the SIVmac239 plasmids at the NotI and NarI sites. The two half plasmids were called Megalo3′ and Megalo5′. Bacteria containing plasmids Megalo3′ and Megalo5′ were deposited on Oct. 11, 2001, at the Collection Nationale de Cultures de Microorganismes (CNCM) at the Institut Pasteur, 25 Rue du Docteur Roux, F-75724, Paris, France under accession numbers I-2728 and I-2729, respectively.

SIVΔNIG and SIVMIG clone 61 constructs. A Nef gene deletion (9500-9670) was engineered into SIVmac239 leaving a SalI site as marker. To do so a XhoI site introduction was first introduced just 3′ to the nef stop codon amplification of two fragments with the following primers Al 5′ GGCGGATCCATAT AGATCTGCGACAGAGACTCTTGCGGG (SEQ ID NO:9) (BglII site underlined) with A3 5′ CCGC CTCGAGTTATTAGCGAGTTTCCTTCTTGTCA (SEQ ID NO:10) (XhoI site underlined) and A2 5′ GCGG CTCGAGAACAGCAGGGACTTTCCACAAGGGG (SEQ ID NO: 11) (BglII site underlined) with A4 5′ GGGCGAATTCCCC GGATCCCTCGACCTGCAGCTGCAAA (SEQ ID NO:12) (BamHI site underlined) in the plasmid. Fragments were purified, digested with XhoI, ligated, digested with BglII and BamHI and ligated into p239SpE3′ devoid of the wild type BglII/BamHI fragment.

The Nef deletion was made by amplification of two fragments amplified using primers A1 with Δnef1 5′ CCGC GTCGACTTACTAGTTATCACAAGAGAGTGAGCTCAAGCCC TTG (SEQ ID NO:13) (SalI site underlined) and A3 with Δnef2 5′ GGCG GTCGACATGTCTCATTTTATAAAAGAA (SEQ ID NO:14) (SalI site underlined). Fragments were purified, digested with SalI, ligated, digested with BglII and XhoI and cloned into the p239SpE3′-XhoI derivative. The complete IRES of encephalomyocarditis virus (EMCV) has been described (3). A 596 bp fragment was amplified using primers I1 5′ GCGC CTCGAGCCCCTCTCCCTCCC (SEQ ID NO: 15) and 12 5′ GTCTCTTGTT CCATGGTTGTGG (SEQ ID NO: 16), Xhol and NcoI underlined. The codon optimised green fluorescent protein (33) was amplified using primers gl 5′ CGCG CCATGGTGAGCAAGGGCGAG (SEQ ID NO:17) (NcoI site underlined) and g2 5′ CCGC CTCGAGTTACTTGTACAGCT (SEQ ID NO:18) (Xhol underlined). The 719 bp GFP fragment was cloned behind the EMCV IRES sequence with the ATG of the GFP gene embedded in the NcoI site. The XhoI-XhoI fragment containing IRES-GFP was cloned into the SalI site in nef deletion. When transfected with the 5′ half plasmid this construct gave rise to a GFP expressing virus called SIVΔNIG. From this half plasmid the Δnef-IRES-eGFP fragment was amplified using primers A1 with B2 5′ GGATC GCGGCCGCTGCTAGGGATTTTCCTGCTTCGG (SEQ ID NO: 19) (NotI site underlined). This fragment was exchanged for BglII/NotI fragment in the 3′ half plasmid (pMT-3). When transfected with the 5′ half plasmid this construct gave rise to a GFP expressing virus called SIVMIG clone 61.

### EXAMPLE 2

### CAT Constructs

Promoter fragments were amplified from the half 5′ plasmids. A fragment spanning the primer binding site to the ATG of the gag gene was amplified from p239SpSp5′ using primers 5′ GGCGCCTGAACAGGGACTTGAAG (SEQ ID NO:20) (NarI site underlined) and 5′ TTTTTTCTCCATCTCCCACTCTATCTTATTACCCCTTCCTG (SEQ ID NO:21) (CAT sequences underlined). CAT and polyA sequences were amplified from an expression plasmid using primers: 5′ GAGTGGGAGATGGAGAAAAAAATCACTGG (SEQ ID NO:22) (CAT sequences underlined) and 5′ ACTAGTGCATGCAGGATCCAGACAT GATAAG (SEQ ID NO:23) (SphI site underlined). The two PCR products were purified and annealed in PCR mix without primers for 5 cycles. External primers were then added for 30 more cycles. Annealed PCR product was cloned, double digested with NarI and SphI, the resulting 1600 bp fragment cloned into pCMV-CAT. A 750 bp HpaI fragment containing the HIV-1 RRE/splice acceptor sequence (25) was added at the SmaI site, just 3′ to the CAT orf. Finally plasmids containing cloned wild type and modified promoter fragments were double digested with NotI and NarI and ligated into the CAT construct. A deleted CMV promoters clone 61 was introduced into the pCMV-CAT plasmid by exchanging NotI/NarI fragments.

All routine cloning was made in the Topo 2.1 TA plasmid (Invitrogen) using Top 10F′ super competent cells (Invitrogen). Sequences of the recombinant viruses are available at ftp.pasteur.fr/pub/retromol.

### EXAMPLE 3

### Transfection and Preparation of Virus Stocks

Half plasmids were double digested with EcoRI and SpeI and ligated. Stocks of SIVmac239, SIVmegalo, SIVmegaloΔTAR, SIVΔNIG or SIVMIG clone 61 were prepared by electroporation of CEMx174 (960 μF, 250V). Virus were harvested at or near the peak of virus production, filtered (0.2 pm), aliquoted and stored at −80° C. Virus preparations were derived from a single passage after transfection on CEMx174 except for SIVΔMC virus which was derived from a 60 day SIVmegalo CEMx174 culture. Titration of infectivity was performed by calculation of the 50% tissue culture infectious dose (TCID₅₀) by the Kärber method and RT concentration was determined by RT assays (Innovagen).

### EXAMPLE 4

### Cell Culture and Virus Replication

CEMx174 lymphoid cells were maintained in RPMI 1640 medium (GIBCO BRL) supplemented with 10% heat-inactivated fetal calf serum (FCS), 1% penicillin (100 U/ml), streptomycin (100 μg/ml). Culture medium was changed twice weekly. PBMCs from healthy, mature rhesus macaques were maintained in RPMI 1640 medium supplemented with 10% heat inactivated FCS, 1% penicillin, streptomycin, 5 μg/ml phytohemagglutinin for the first two days after which 2000 U/ml human recombinant IL-2 and 50% MLA 144 supernatant were added for the remainder. Infections were performed on 5×106 cells in 100 μl of virus stock during 2 hours at 37° C. then cells were washed twice and resuspended in 5 ml of culture medium. RT activity was determined on 10 μl centrifuge supernatant as recommended (Invitrogen). All CEMx174 timepoints were made in triplicate.

### EXAMPLE 5

### Sequence Analyses of Recombinants Viruses

Total CEMx174 or macaque PBMC genomic DNA was extracted using Masterpure extraction kit (Epicentre). Chimeric or wild type LTR DNA were nested amplified under standard conditions using flanking primers i.e. 5′ CTAACCGCAAGAGGCCTTCTTAACATG (SEQ ID NO:24) and 5′ GGAGTCACTCTGCCCAGCACCGGCCCA (SEQ ID NO:25) then 5′ GGCTGACAAGAAGGAAACTCGCTA (SEQ ID NO:26) and 5′ GGAGTCACTCTGCCCAGCACCGGCCAAG (SEQ ID NO:27). Products were cloned using the Topo 2.1 TA and sequenced using an Applied Biosystems 373A DNA sequencer. Sequencing primers were 5′ ATGGAAAACCCAGCTGAAG (SEQ ID NO:28), 5′ CCCAGTACATGACCTTATGGG (SEQ ID NO:29), 5′ CCAAAACCGCATCACCATGG (SEQ ID NO:30) and 5′ TCTTCCCTGA CAAGACGGAG (SEQ ID NO:31).

### EXAMPLE 6

### CAT Assays

HIV-1 Tat and Rev expressing plasmids, pSV2/Tat HIV and pBLSV/Rev have been described (23, 26). For each assay 4×106 CEMx174 were transfected with 8 μg of CAT plasmid and 3 μg of pBLSV/Rev HIV with or without 3 μg pSV2/Tat expression plasmids using the DEAE-dextran method. When pSV2/Tat was not used 3 μg of pSV2gpt was added. After 4 days, the concentration of total protein lysates was determined by a commercial dye-binding method (Bio-Rad) and equal amounts of protein were used in standard CAT assays. All experiments were conduced at least twice including pAIIIR plasmid (35) as a positive control and pSV2gpt as negative control. Chromatograms were quantified using a Molecular Dynamics phosphor imager. Relative conversion was determined by normalizing the amount of acetylated C14 chloramphenicol of mutants constructions with respect to the SIVmac239 promoter activity in the presence of Tat control multiplied by 100.

### EXAMPLE 7

### SIV Inoculation

Rhesus monkeys (Macaca mulatta) of Chinese origin were serologically negative for SIV, type D retrovirus and simian foamy virus. Animals were inoculated intravenously with 200 TCID₅₀ of SIVmac239, SIVmegalo and SIVΔMC. Blood and serum samples were drawn twice weekly during the first month, once a week during the two following months.

### EXAMPLE 8

### SIV Quantitation and Antibody Titration

SIV serum titres were quantified by bDNA signal amplification (Bayer, Amsterdam). The cut off was 400 viral RNA copies/ml of serum for 1 ml tested. Antibody titres were determined using the Sanofi-Pasteur kit.

### EXAMPLE 9

### In Situ Hybridization (ISH)

In situ hybridization was performed on frozen lymph node mononuclear cells (LNMC) as previously described with a 35S-labeled SIVmac142 env-nef RNA probe (5).

### EXAMPLE 10

### Replication of Chimeric SIV-CMV Promoter Constructs on CEMx174

The SIV U3 promoter sequences following the Nef stop codon were replaced by those of the powerful immediate early 2 promoter from human CMV. Two constructs were made differing only in the presence or absence of SIV TAR sequences (FIG. 1). For SIVmegaloΔTAR the double TAR stem-loop motifs were deleted (1 to 93). In this case the transcription start site of the CMV-IE promoter was retained along with the first 59 bp downstream. All the recombinant plasmids were checked by sequencing.

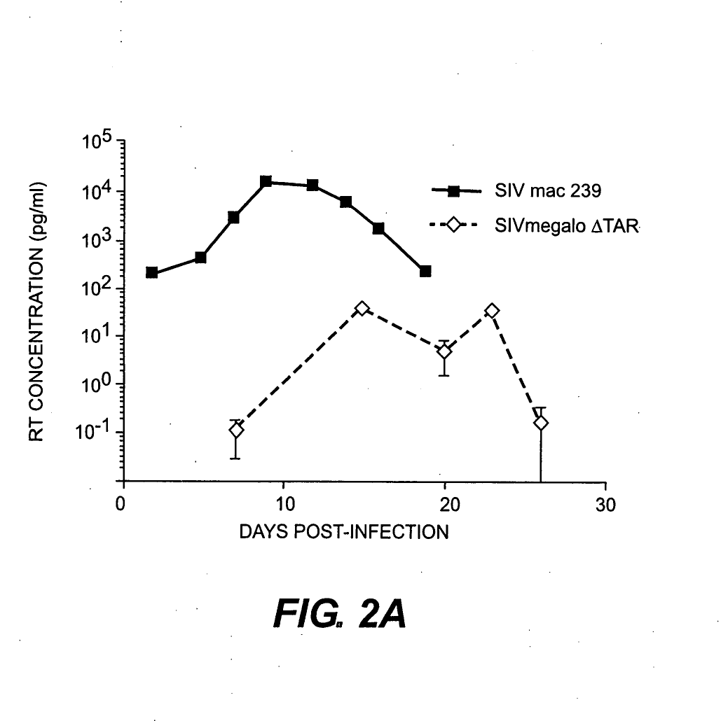

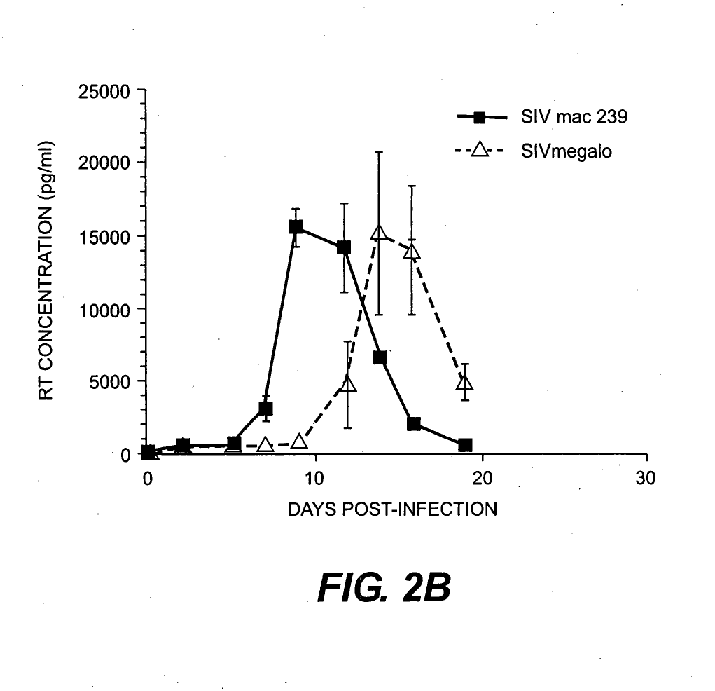

CEMx174 cells were transfected with ligated inserts derived from half plasmids. Supernatants were harvested regularly and viral stocks made when RT activity was maximal. For replication studies, five million CEMx174 cells were infected with 1 ng of RT activity which corresponds to ˜1 TCID₅₀ per 10³ cells, except for SIVmegaloΔTAR for which it was impossible to obtain more than 0.1 ng/ml of RT activity. SIVmegalΔTAR grew very poorly with a peak viremia approximately 3 logs lower than SIVmac239 and delayed by 10 days (FIG. 2A). Not surprisingly, no cytopathic effect was observed. By contrast peak viremia of SIVmegalo was comparable to that of SIVmac239 although the peak was delayed by a week (FIG. 2B) and no difference could be observed compared to wild type virus in terms of virus cytopathogenicity or the morphology of viral particles as seen by electronic microscopy (not shown).

In order to understand the delayed peak viremia for SIVmegalo, the promoter region was analyzed to verify its stability. Primers spanning the cloning sites were used to amplify the promoter region from total cellular DNA from SIVmegalo infected CEMx174 cells. Of three independent cultures, a typical analysis is shown in FIG. 3A. Deletions in the promoter were apparent as early as day 6, while by day 15 most amplicons harboured deletions. Samples at day 15 and 60 were cloned and sequenced. Most samples collected 15 days after culture on CEMx174 showed a promoter distal deletions in the region −420 to −130 bp (FIG. 3B). Many involved deletions between the numerous 17, 18, 19 and 21 bp repeats sequences in the CMV-IE promoter, although there were deletions elsewhere. By day 60, one promoter form dominated the culture. It resulted from a 269 bp deletion between the second and forth 19 bp repeats which harbour CRE sites (FIG. 3B). A few point mutations were observed in the promoter or TAR sequences although they never went to fixation. A stock virus, named SIVΔMC, was derived after 60 days of culture on CEMx174. When this stock was used to infect CEMx174 cells it grew as well as the parental SIVmac239 virus (FIG. 3C).

### EXAMPLE 11

### Chimeric Promoter Activity

Promoter activities were analyzed in standard CAT assays. Transcriptional activities were determined using CAT reporter gene cloned in exactly the position of the gag. In order to avoid irrelevant splicing HIV-1 RRE sequence was added downstream of CAT at the HpaI site (FIG. 4A). Conversion was normalised to the wild type activity in the presence of HIV-1 Tat and Rev protein known to act in trans on SIV sequences (12, 23). Although as expected, the SIVmegaloΔTAR could not be transactivated by Tat (FIG. 4B), basal transcription was comparable to that of SIVmac239 in the absence of Tat. For SIVmegalo, a 70% reduction of Tat transactivated promoter activity compared to SIVmac239 promoter was noted indicating that the promoter was not as powerful despite encoding two NF-kB and three Sp1 sites in the promoter proximal region. The variant promoter from the SIVΔMC, clone 61, was subcloned and analysed in a CAT assay. This clone performed a little better than wild type virus and was stronger than the SIVmegalo promoter which helps explain why it started to outgrow the parental virus after 15 days in CEMx174 culture.

### EXAMPLE 12

### Replication of Chimeric Viruses on Macaque PBMCs

SIVmegalo and SIVmac239 were used to infect PHA-stimulated PBMCs from three naive rhesus monkeys in the presence of human interleukin 2. The equivalent of 1 ng of RT activity was used to infect 5×106 PBMCs. SIVmegalo replication was delayed by 4 to 10 days compared to wild type virus (FIGS. 5A and D). For all cultures, deletions in the SIVmegalo promoter were noted by 10-15 days post infection (FIG. 5A). A heterogeneous collection of promoters were found in the 30 day PBMC sample (FIG. 5B). Most harboured deletions in the same region of the CMV-IE promoter between −450 and −200 bp although a few promoter proximal deletions were apparent. The replication of SIVmegalo on other macaques PBMCs shows the virus grow poorly whereas SIVΔMC grow to wild type titers although with slightly delayed Kinetics (FIG. 5D).

### EXAMPLE 13

### In Vivo Studies

Two rhesus macaques (93029 and 93035) were inoculated intravenously with 200 TCID₅₀ of SIVmegalo. Viral replication was tested by bDNA Chiron test. The virus replicated very poorly indeed with only one serum sample scoring positive (6K copies/ml) for viral RNA, and this at day 4 (FIG. 6A). All other timepoints out to day 100 proved negative. However, antibody titres started to come up by 30-45 days post infection (FIG. 6B) suggesting that the animals were infected. This was confirmed highly sensitive amplification (nested env V1-V2, sensitivity 1-2 copies per reaction (7)) of proviral DNA from PBMCs (FIG. 6C). Even so, detection was intermittent suggesting that the titres were low and around the threshold of detection, i.e. 1/200,000 cells. Moreover, in situ hybridisation failed to detect any productively infected cells in lymph node mononuclear cells (LNMC) one hundred days after infection in SwVmegalo infected macaque (not shown). Moreover CD4 count were stable throughout the course of primary infection (not shown). Two rhesus monkeys (Macacca mulatta) were infected with 200 TCID50 of a SIVmegalo virus stock. For animal 93035 there was hardly any viremia at all, just one point at 6000 RNA copies per ml at day 4 and thereafter nothing for out to one year. The test used was the Bayer bDNA method with a cut-off of 400 copies/ml. PCR on DNA extracted from peripheral blood mononuclear cells (PBMCs) showed that SIV proviral DNA could be occasionally found, in fact 14/43 attempts. This indicates that despite growing extremely poorly, the virus was able to persist. For this animal, antibody titres started coming up by two months and plateaued by six months. The antibody ELISA titres at plateau were a factor of 10 to 100 down on what is normally observed in macaques infected by the reference strain SIVmac239.

The second animal (no. 93029) was inoculated with the same dose of SIVmegallo. No virus whatsoever could be detected in the periphery by the bDNA assay, as though there the virus had not taken. Followed the animal for 6 months showed that antibody came up and plateaued by 3 months indicative of infection. SIV proviral DNA could be detected in PBMCs intermittently (18/26 attempts) confirming that the animal had truly been infected.

A variant of the SIVmegallo virus, termed SIVΔMC, was constructed which contained a ˜270 bp deletion within the CMV-IE promoter (see FIG. 7). Macaque 94025 was infected by SIVΔMC with the same dose that for SIVmegalo. There was a small peak of viremia (25,700 copies/ml) at 30 days p.i. after which viremia was undetectable, i.e., <400 copies/ml (FIG. 6A). Antibody was detectable by 20 days p.i., earlier than for SIVmegalo (FIG. 6B). Like the SIVmegallo infected animals, antibody titres plateaued by three months post infection and plateaued at a level 10 to 100 fold lower than SIVmac239 infected animals. They remained steady for 9 months. Amplification of proviral DNA from PBMCs showed that the virus had persisted (FIG. 6C).

As controls two animals (960548 and 960836) were infected intravenously with the same dose that for SIVmegalo and SIVΔMC of SIVmac239. Peak viremia was in excess of 100K copies/ml (FIG. 6A) while a high titre antibody response was already detectable by day 20 p.i. (FIG. 6B) and proviral DNA was detectable from day four (6C).

To check the stability of the SIVmegalo promoter the region was amplified from DNA extracted from a lymph node from SIVmegalo infected monkey (93035) taken at day 100. Viruses in the lymph node sample all had the same 190 bp deletion in the 5′ enhancer region (FIG. 7), which corresponds to a deletion noted in infected PBMCs from macaque 93035 (FIG. 5C). Moreover, trivial sequence variation among these clones suggested that this virus was replicating (not shown).

### EXAMPLE 14

### Challenge by SIVmac239

All three animals (93035, 93029 & 94025) were challenged by the intravenous route with 200 TCID₅₀ of a standard stock of SIVmac239. This is equivalent to ˜2000 AID₅₀ (animal infectious doses). Normally 1 TCID50 is enough to infect animals. As controls two naive animals (nos 960548 & 960836) were inoculated SIVmac239. Both showed signs of high primary viremia by day 15 which is perfectly normal. Viremia then settled down to a titre of around 105/ml. High ELISA titre antibody was elicited within one month of infection. These findings confirm that the challenge stock was behaving in our hands as expected.

Challenge of the three animals already infected by SIVmegallo or SIVΔMC failed to breakthrough. No detectable plasma viremia (i.e. <400 copies/ml) was found at all timepoints out to two months post-challenge.

The inoculating viruses (SIVmegalo and SIVΔMC) and the challenge viruses (SIVmac239) differ only in their LTRs, notably their size. Therefore in order to ascertain whether the challenge 239 virus took in the animals a fragment spanning the U3 promoter region was amplified with oligos common to the inoculating and challenge virus. The size of the corresponding fragment from SIVmac239 challenge virus is 260 bp, while those of SIVmegalo and SIVΔMC are 657 and 386 bp respectively. Hence amplification of this region could distinguish the three viruses.

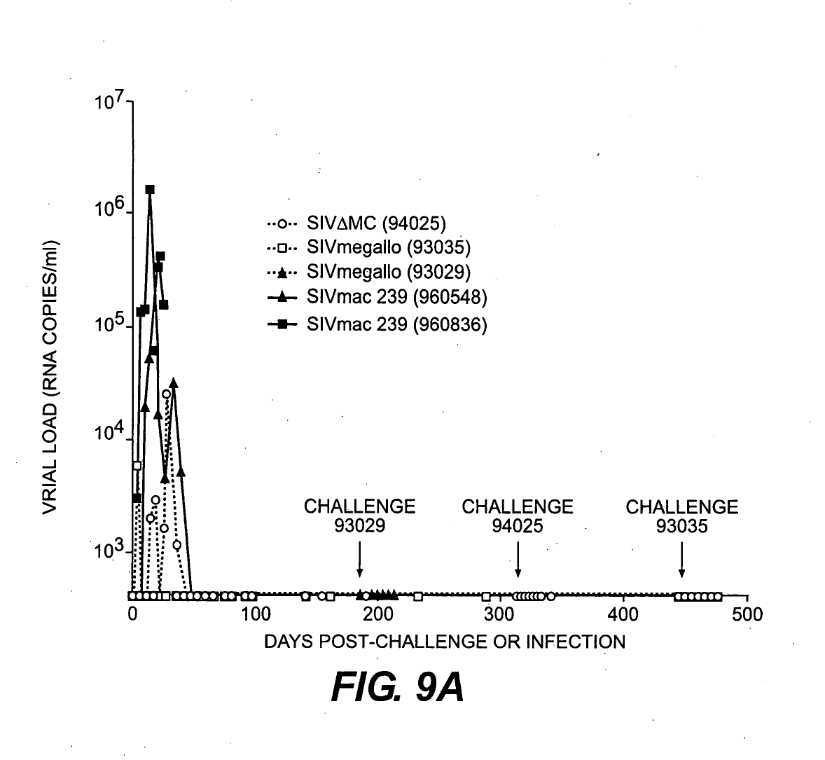

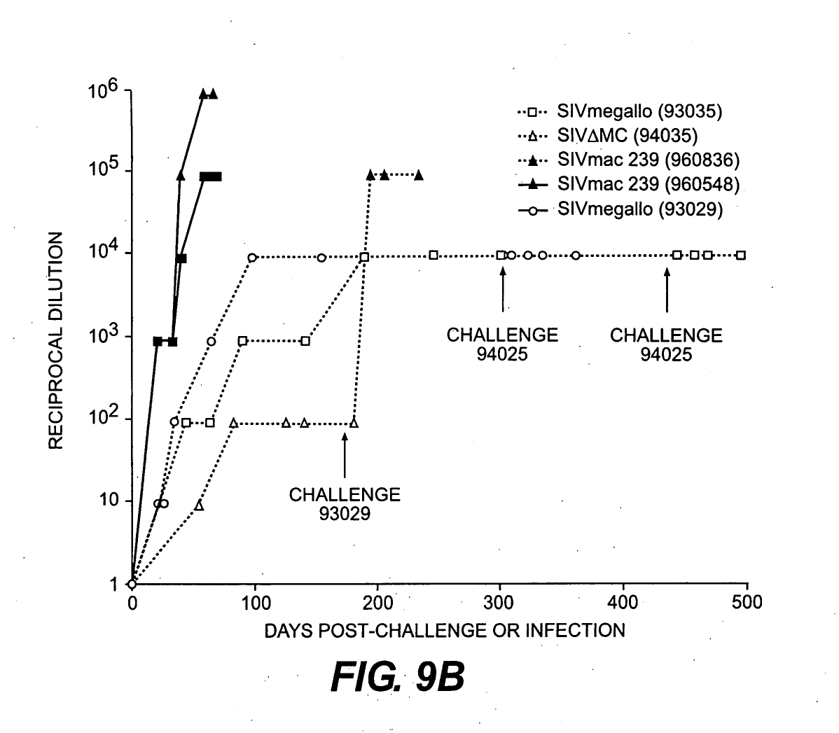

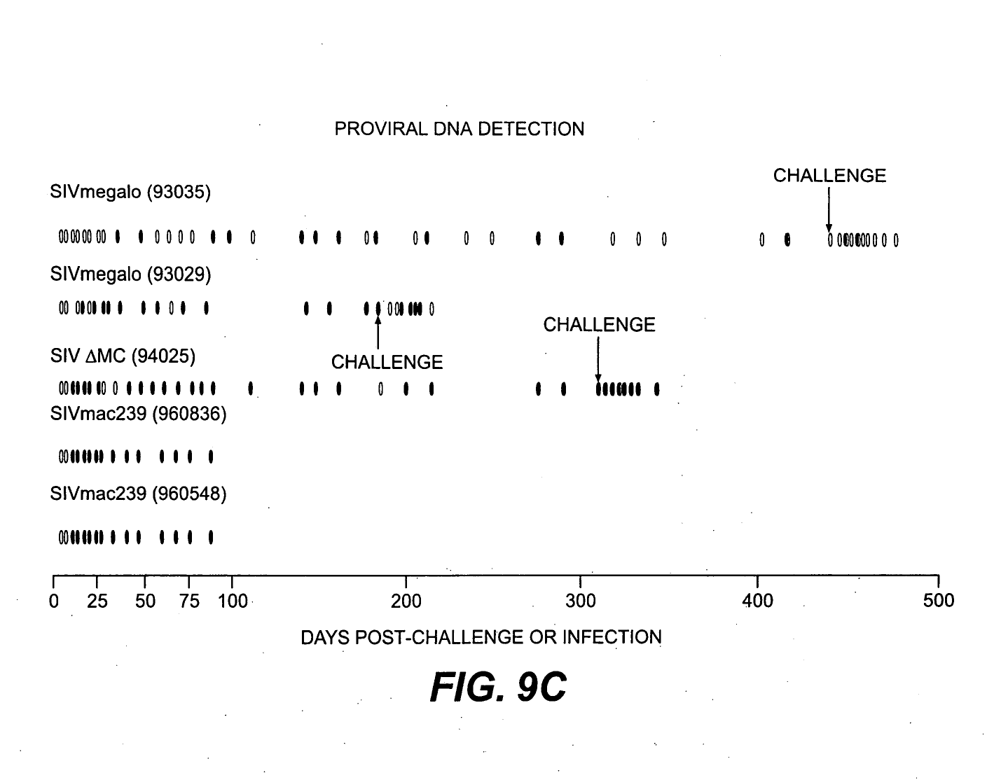

As can be seen from FIG. 9, the challenge virus could be recovered from all three animals although plasma viremia was negative. For macaque 93029, inoculated by SIVmegalo, there was a 2 log boost in the anti-SIV ELISA titres by suggestive of SIVmac239 replication. However for the other two animals, where the anti-SIV ELISA titres were much greater than for 93029, there was no detectable increase in titre over 2 months of follow-up suggesting that replication of the challenge virus was strongly curtailed.

### EXAMPLE 15

### GFP Constructs

Clearly SIVmegalo grew very poorly in vivo (FIG. 6A) in contrast to what was observed ex vivo (FIGS. 3C and 5A). As the CMV-IE promoter is expressed in a wide variety of cells, it might be supposed that the virus is more transcriptionally active in non-activated cells than parental SIVmac239 which would make it particularly vulnerable to cell mediated immunity in vivo.

To test this notion, the nef gene in wild type virus or in SIVΔMC clone 61 virus was replaced by with the IRES-eGFP reporter gene (FIG. 8A). Stocks of these viruses, termed SIVΔNIG and SIVMIG clone 61 respectively, were made on CEMx174 cells. Fluorescent microscopy confirmed the expression of the eGFP gene in CEMx174 (FIG. 8B). Macaque PBMCs were isolated and directly infected with either SIVΔNIG or SIVMIG clone 61. Although low gfp fluorescence was obtained (0,7- 2,1%), no significant differences in mean eGFP fluorescence per cell were noted for the two viruses (FIG. 8B).

### EXAMPLE 16

### SIVΔMC Constructs

A promoter fragment derived from 60 day culture of SIVmegalo on CEMX174 cells was cloned in place of the CMV-IE insert in plasmids Megalo5′ and Megalo3′. The two half plasmids were called ΔMC3′ (or delta MC3′) and ΔMC5′ (or delta MC5′). Bacteria containing plasmids ΔMC3′ and ΔMC5′ were deposited on Oct. 11, 2001, at the Collection Nationale de Cultures de Microorganismes (CNCM) at the Institut Pasteur, 25 Rue du Docteur Roux, F-75724, Paris, France under accession numbers I-2726 and I-2727, respectively.

The SIV ΔMC3′ (or SIV delta MC3′) and SIV ΔMC5′ (or SIV delta MC5′ plasmids contain the following promoter sequence:

| | |
|---|---|
| 5′ GCTAAAAGCGGCCGCTTACATAACTTACGGTAA | (SEQ ID NO:32) |
| ATGGCCCGCCTGGCTGACCGCCCAACGACCCCCGCC | |
| CATTGACGTCAATAATGACGTATGTTCCCATAGTAA | |
| CGCCAATAGGGACTTTCCATTGACGTCAATGGGTGT | |
| TTGTTTTGGCACCAAAATCAACGGGACTTTCCAAAA | |
| TGTCGTAACAACTCCGCCCCATTGACGCAAATGGGC | |
| GGTAGGCGTGTACGGTGGGAGGTCTATATAAGCAGA | |
| GCTCGTTTAGTGAACCGTCAGTCGCT-3′. | |

### REFERENCES

1. Addo, M., M. Altfeld, E. Rosenberg, R. Eldridge, M. Philips, K. Habeeb, A. Khatri, C. Brander, G. Robbins, G. Mazzara, G. P J, and B. Walker. 2001. The HIV-1 regulatory proteins Tat and Rev are frequently targeted by cytotoxic T lymphocytes derived from HIV-1-infected individuals. Proc. Natl. Acad. Sci. U.S.A. 98:1781-6.

2. Amara, R. R., F. Villinger, J. D. Altman, S. L. Lydy, S. P. O'Neil, S. I. Staprans, D. C. Montefiori, Y. Xu, J. G. Herndon, L. S. Wyatt, M. A. Candido, N. L. Kozyr, P. L. Earl, J. M. Smith, H. L. Ma, B. D. Grimm, M. L. Hulsey, J. Miller, H. M. McClure, J. M. McNicholl, B. Moss, and H. L. Robinson. 2001. Control of a mucosal challenge and prevention of AIDS by a multiprotein DNA/MVA vaccine. Science. 292:69-74.

3. Borman, A., P. Le Mercier, M. Girard, and K. Kean. 1997. Comparison of picomaviral IRES-driven internal initiation of translation in cultured cells of different origins. Nucleic Acids Res. 25:925-32.

4. Cafaro, A., A. Caputo, C. Fracasso, M. Maggiorella, D. Goletti, S. Baroncelli, M. Pace, L. Semicola, M. Koanga-Mogtomo, M. Betti, A. Borsetti, R. Belli, L. Akerblom, F. Corrias, S. Butto, J. Heeney, P. Verani, F. Titti, and B. Ensoli. 1999. Control of SHIV-89.6P-infection of cynomolgus monkeys by HIV-1 Tat protein vaccine. Nat. Med. 5:643-50.

5. Chakrabarti, L., M. C. Cumont, L. Montagnier, and B. Hurtrel. 1994. Kinetics of primary SIV infection in lymph nodes. J. Med. Primatol. 23:117-124.

6. Chang, L., E. McNulty, and M. Martin. 1993. Human immunodeficiency viruses containing heterologous enhancer/promoters are replication competent and exhibit different lymphocyte tropisms. J. Virol. 67:743-52.

7. Cheynier, R., S. Gratton, M. Halloran, I. Stahmer, N. L. Letvin, and S. Wain-Hobson. 1998. Antigenic stimulation by BCG vaccine as an in vivo driving force for SIV replication and dissemination. Nat. Med. 4:421-7.

8. Cheynier, R., S. Henrichwark, F. Hadida, E. Pelletier, E. Oksenhendler, B. Autran, and S. Wain-Hobson. 1994. HIV and T cell expansion in splenic white pulps is accompanied by infiltration of HIV-specific cytotoxic T lymphocytes. Cell. 78:373-87.

9. Chun, T. W., and A. S. Fauci. 1999. Latent reservoirs of HIV: obstacles to the eradication of virus. Proc. Natl. Acad. Sci. U.S.A. 96:10958-61.

10. Daniel, M. D.,F. Kirchhoff, S. C. Czajak, P. K. Sehgal, and R. C. Desrosiers. 1992. Protective effects of a live attenuated SIV vaccine with a deletion in the nef gene. Science. 258:1938-41.

11. Desrosiers, R., J. Lifson, J. Gibbs, S. Czajak, A. Howe, L. Arthur, and R. Johnson. 1998. Identification of highly attenuated mutants of simian immunodeficiency virus. J. Virol. 72:1431-7.

12. Emerman, M., M. Guyader, L. Montagnier, D. Baltimore, and M. Muesing. 1987. The specificity of the human immunodeficiency virus type 2 transactivator is different from that of human immunodeficiency virus type 1. EMBO J. 6:3755-60.

13. Gorelick, R. J., R. E. Benveniste, J. D. Lifson, J. L. Yovandich, W. R. Morton, L. Kuller, B. M. Flynn, B. A. Fisher, J. L. Rossio, M. Piatak, Jr., J. W. Bess, Jr., L. E. Henderson, and L. O. Arthur. 2000. Protection of Macaca nemestrina from disease following pathogenic simian immunodeficiency virus (SIV) challenge: utilization of SIV nucleocapsid mutant DNA vaccines with and without an SIV protein boost. J. Virol. 74:11935-49.

14. Greenough, T., J. Sullivan, and R. Desrosiers. 1996. Declining CD4 T-cell counts in a person infected with nef-deleted HIV-1. N. Engl. J. Med. 340:236-7.

15. Guan, Y., J. Whitney, M. Detorio, and M. Wainberg. 2001. Construction and in vitro properties of a series of attenuated simian immunodeficiency viruses with all accessory genes deleted. J. Virol. 75:4056-67.

16. Gundlach, B. R., M. G. Lewis, S. Sopper, T. Schnell, J. Sodroski, C. Stahl-Hennig, and K. Uberla. 2000. Evidence for recombination of live, attenuated immunodeficiency virus vaccine with challenge virus to a more virulent strain. J. Virol. 74:3537-42.

17. Ilyinskii, P., M. Simon, S. Czajak, A. Lackner, and R. Desrosiers. 1997. Induction of AIDS by simian immunodeficiency virus lacking NF-kappaB and Sp1 binding elements. J. Virol. 71:1880-7.

18. Ilyinskii, P. O., and R. Desrosiers. 1996. Efficient transcription and replication of simian immunodeficiency virus in the absence of NF-KB and Sp1 binding element. J. Virol. 70:3118-3126.

19. Johnson, R., J. Lifson, S. Czajak, K. Cole, K. Manson, R. Glickman, J. Yang, D. Montefiori, R. Montelaro, M. Wyand, and R. Desrosiers. 1999. Highly attenuated vaccine strains of simian immunodeficiency virus protect against vaginal challenge: inverse relationship of degree of protection with level of attenuation. J. Virol. 73:4952-61.

20. Kestler, H., T. Kodama, D. Regier, P. Sehgal, M. Daniel, N. King, and R. C. Desrosiers. 1990. Induction of AIDS in rhesus monkeys by molecularly cloned simian immunodeficiency virus. Science. 248:1109-1112.

21. Kestler, H. W. I., D. J. Ringler, K. Mori, D. L. Panicali, P. K. Sehgal, M. D. Daniel, and R. C. Desrosiers. 1991. Importance of the nef gene for maintenance of high virus load and for development of AIDS. Cell. 65:651-662.

22. Khatissian, E., V. Monceaux, M. Cumont, M. Kieny, A. Aubertin, and B. Hurtrel. 2001. Persistence of pathogenic challenge virus in macaques protected by simian immunodeficiency virus SIVmacDeltanef. J. Virol. 75:1507-15.

23. Malim, M., S. Bohnlein, R. Fenrick, S. Le, J. Maizel, and B. Cullen. 1989. Functional comparison of the Rev trans-activators encoded by different primate immunodeficiency virus species. Proc. Natl. Acad. Sci. U S A. 86:8222-6.

24. Mansky, L. M., and H. M. Temin. 1995. Lower in vivo mutation rate of human immunodeficiency virus type 1 than that predicted from the fidelity of purified reverse transcriptase. J. Virol. 69:5087-5094.

25. Martins, L., N. Chenciner, B. Asjo, A. Meyerhans, and S. Wain-Hobson. 1991. Independent fluctuation of human immunodeficiency virus type 1 rev and gp41 quasispecies in vivo. J. Virol. 65:4502-7.

26. Meyerhans, A., R. Cheynier, J. Albert, M. Seth, S. Kwok, J. J. Sninsky, L. Morfeldt-Manson, B. Åsjö, and S. Wain-Hobson. 1989. Temporal fluctuations in HIV quasispecies in vivo are not reflected by sequential HIV isolation Cell. 58:901-910.

27. Mocarski, E. 1996. Cytomegalovirus and their replication Fields Virology. 3rd edition:2447-92.

28. Osterhaus, A., C. van Baalen, R. Gruters, M. Schutten, C. Siebelink, E. Hulskotte, E. Tijhaar, R. Randall, G. van Amerongen, A. Fleuchaus, V. Erfle, and G. Sutter. 1999. Vaccination with Rev and Tat against AIDS. Vaccine. 17:2713-4.

29. Ostrowski, M. A., D. C. Krakauer, Y. Li, S. J. Justement, G. Learn, L. A. Ehler, S. K. Stanley, M. Nowak, and A. S. Fauci. 1998. Effect of immune activation on the dynamics of human immunodeficiency virus replication and on the distribution of viral quasispecies. J. Virol. 72:7772-84.

30. Pauza, C., P. Trivedi, M. Wallace, T. Ruckwardt, H. Le Buanec, W. Lu, B. Bizzini, A. Burny, D. Zagury, and R. Gallo. 2000. Vaccination with tat toxoid attenuates disease in simian/HIV-challenged macaques. Proc. Natl. Acad. Sci. U.S.A. 97:3515-9.

31. Pezo, V., and S. Wain-Hobson. 1997. Dynamics of HIV mutation in vivo. Journal of Infection. 34:201-203.

32. Pohlmann, S., S. Floss, P. Ilyinskii, T. Stamminger, and F. Kirchhoff. 1998. Sequences just upstream of the simian immunodeficiency virus core enhancer allow efficient replication in the absence of NF-kappaB and Sp1 binding elements. J. Virol. 72:5589-98.

33. Prasher, D. 1995. Using GFP to see the light Trends Genet. 11(8):320-3.

34. Regier, D., and R. Desrosiers. 1990. The complete nucleotide sequence of a pathogenic molecular clone of simian immunodeficiency virus AIDS Res. Hum. Retroviruses. 6:1221-31.

35. Rosen, C., E. Terwilliger, A. Dayton, J. Sodroski, and W. Haseltine. 1988. Intragenic cis-acting art gene-responsive sequences of the human immunodeficiency virus. Proc. Natl. Acad. Sci. U S A. 85:2071-5.

36. Stinski, M., and T. Roehr. 1985. Activation of the major immediate early gene of human cytomegalovirus by cis-acting elements in the promoter-regulatory sequence and by virus-specific trans-acting components. J. Virol. 55:431-41.

37. Switzer, W., S. Wiktor, V. Soriano, A. Silva-Graca, K. Mansinho, I. Coulibaly, E. Ekpini, A. Greenberg, T. Folks, and W. Heneine 1998. Evidence of Nef truncation in human immunodeficiency virus type 2 infection J. Infect. Dis. 177(1):65-71.

38. Westrop, G., K. Wareham, D. Evans, G. Dunn, P. Minor, D. Magrath, F. Taffs, S. Marsden, M. Skinner, G. Schild, and e. al 1989. Genetic basis of attenuation of the Sabin type 3 oral poliovirus vaccine J. Virol. 63(3):1338-44.

39. Wyand, M., K. Manson, M. Garcia-Moll, D. Montefiori, and R. Desrosiers 1996. Vaccine protection by a triple deletion mutant of simian immunodeficiency virus J. Virol. 70:3724-3733.

40. Sirven, A., Ravet, E., Charneau, P., Zennou, V., Coulombel, L., Guetard, D., Pflumio, F., and Dubart-Kupperschmitt, A. (2001). Enhanced transgene expression in cord blood CD34(+)-derived hematopoietic cells, including developing T cells and NOD/SCID mouse repopulating cells, following transduction with modified trip lentiviral vectors, Mol Ther 3, 438-48.

1. A recombinant HIV virus comprising replacement sequences comprising heterologous transcriptional regulatory elements replacing natural transcriptional regulatory elements in the U3 region of the virus, wherein the virus has decreased replication in vivo and the virus has a protective effect when administered to a host.

2-40. (canceled)
